(19) United States
(12) Patent Application Publication
(10) Pub. No.: US 2026/0130901 A1
(43) Pub. Date: May 14, 2026

(54) CGRP RECEPTOR ANTAGONIST FOR THE TREATMENT OF MIGRAINE

(71) Applicant: ALLERGAN PHARMACEUTICALS INTERNATIONAL LIMITED, Dublin 17 (IE)

(72) Inventor: Joel M. Trugman, Hoboken, NJ (US)

(21) Appl. No.: 19/119,953

(22) PCT Filed: Oct. 11, 2023
(86) PCT No.: PCT/US2023/076576

§ 371 Date: Apr. 10, 2025

Related U.S. Application Data
(60) Provisional application No. 63/379,075, filed on Oct. 11, 2022.
(60) Provisional application No. 63/490,918, filed on Mar. 17, 2023.
(60) Provisional application No. 63/522,501, filed on Jun. 22, 2023.

(51) Int. Cl. A61K 31/4545; A61K 31/496; A61P 25/06

(52) U.S. Cl. CPC A61K 31/4545; A61K 31/496; A61P 25/06

(57) ABSTRACT

The present disclosure provides methods for the treatment of migraine, such as the acute treatment of migraine, in particular during the prodrome stage of a migraine attack, comprising administering a CGRP receptor antagonist. In particular, the present disclosure provides methods for administering ubrogepant during the prodrome or premonitory phase for the acute treatment of migraine.

### RELATED APPLICATIONS

This application claims the benefit of priority to U.S. Provisional Patent Application No. 63/379,075, filed Oct. 11, 2022; U.S. Provisional Patent Application No. 63/490,918, filed Mar. 17, 2023; and U.S. Provisional Patent Application No. 63/522,501, filed Jun. 22, 2023; the entire contents of each of which are incorporated herein by reference.

### FIELD

The present disclosure is related to medicaments and methods for treating migraine. In particular, the present disclosure is related to medicaments and methods for the acute treatment of migraine during the prodrome stage of a migraine attack.

### BACKGROUND

Migraine is a highly prevalent, severe, and disabling neurological condition with a significant unmet need for effective treatments. (Holland, P. R. & Goadsby, P. J. Neurotherapeutics (2018). Migraine affects over 1 billion people worldwide, and it was reported as the second leading cause of disability in the 2016 Global Burden of Disease study. See GBD 2019 Diseases and Injuries Collaborators. Global Burden of 369 diseases and injuries in 204 countries and territories, 1990-2019: a systemic analysis for the Global Burden of Disease Study 2019, Lancet 2020; 396:1204-22.

Treatment guidelines from the American Headache Society recommend treating acute migraine as early as possible after the onset of headache pain, as delayed treatment may increase pain severity and disability. See Marmura M J1, Silberstein S D, Schwedt T J. The acute treatment of migraine in adults: The American Headache Society Evidence Assessment of Migraine Pharmacotherapies. Headache. 2015 January; 55(1):3-20. However, treating a migraine at the beginning of the headache phase may still be a suboptimal treatment for patients, as patients must still contend with both headache and nonheadache migraine symptoms.

There remains a need to develop effective methods for the acute treatment of migraine.

### SUMMARY

The present disclosure provides methods for treating migraine, such as methods for the acute treatment of migraine, during the prodrome stage of a migraine.

The present disclosure provides methods for the treatment of migraine, comprising administering a CGRP receptor antagonist to a patient in need thereof, wherein the CGRP receptor antagonist is administered during the prodrome stage of a migraine attack. In embodiments, the CGRP receptor antagonist is selected from the group consisting of ubrogepant, atogepant, rimegepant, and zavegepant, or a pharmaceutically acceptable salt thereof. Administration of the CGRP receptor antagonist during the prodrome stage of a migraine attack (e.g., before the patient is experiencing a migraine headache) may reduce the severity of or eliminate a migraine headache.

The present disclosure provides methods of treating migraine, comprising administering ubrogepant or a pharmaceutically acceptable salt thereof to a patient in need thereof, wherein ubrogepant is administered during the prodrome stage of a migraine attack. In embodiments, administration of ubrogepant results in a statistically significant treatment effect with respect to the absence of a headache of moderate or severe intensity within 24 hours or within 48 hours of administration as compared to placebo. In embodiments, administration of ubrogepant results in a statistically significant treatment effect with respect to the absence of a headache of any intensity within 24 hours of administration as compared to placebo.

### BRIEF DESCRIPTION OF THE DRAWINGS

FIG. 1 shows the study design of a phase 3, multicenter, randomized, double-blind, placebo-controlled crossover study carried out to evaluate the efficacy, safety, and tolerability of oral ubrogepant for the acute treatment of migraine when administered during the prodrome. The results of the study show that treatment during the initial phase of a migraine attack (i.e., the prodrome or premonitory phase), prior to the onset of headache, prevents the development of moderate/severe headache, or headache of any intensity, over the next 24 hours. In addition, when administered during the prodrome, ubrogepant improves the ability to function normally over the next 24 hours.

FIG. 2 shows participant flow in the phase 3 study evaluating the efficacy, safety, and tolerability of oral ubrogepant for the acute treatment of migraine when administered during the prodrome.

FIG. 3 depicts criteria for determination of responders and nonresponders on the primary efficacy endpoint: Absence of Moderate/Severe Headache within 24 Hours.

FIG. 4 shows the severity of the most common prodrome symptoms reported during the study.

FIG. 5 shows the difference in response rates with respect to absence of sensitivity to light for 100 mg ubrogepant and placebo. The proportion of events with absence of sensitivity to light after treatment with ubrogepant 100 mg administered during prodrome was numerically greater than placebo, starting at 2 hours post-dose and extending through 48 hours.

FIG. 6 shows the difference in response rate for absence of fatigue for ubrogepant 100 mg and placebo. The difference in response rates for the ubrogepant 100 mg dose group compared with placebo as early as 3 hours post-dose administered during the prodrome.

FIG. 7 shows the difference in response rates for absence of neck pain over time for ubrogepant 100 mg and placebo. The difference in response rates was nominally significant for the ubrogepant 100 mg dose group compared with placebo as early as 3 hours post-dose administered during the prodrome.

FIG. 8 shows the difference in response rates for absence of sensitivity to sound for ubrogepant 100 mg and placebo. Numerically higher response rates for the ubrogepant 100 mg dose group compared with placebo administered during the prodrome were observed.

FIG. 9 shows the difference in response rates for absence of dizziness over time for ubrogepant 100 mg and placebo. Numerically higher response rates for the ubrogepant 100 mg dose group compared with placebo administered during the prodrome were observed.

FIG. 10 shows results for ability to function normally over 24 hours in the mITT population, which demonstrated statistically significant treatment differences between ubrogepant and placebo on ability to function normally over 48 hours.

FIG. 11 shows the proportion of participants treated with ubrogepant who reported being “satisfied” or “extremely satisfied” with treatment compared with placebo at 8 hours and 24 hours post-dose.

FIG. 12 shows proportion of participants with absence of moderate/severe headache within 24 hours and 48 hours and absence of any intensity headache within 24 hours after treatment during the prodrome.

### DETAILED DESCRIPTION

Migraine is a multiphasic, episodic disease of the nervous system with four generally recognized phases, each with a unique biology: prodrome/premonitory phase, aura phase, headache phase, and post-drome/postheadache phase. See Dodick D W, “A Phase-by-Phase Review of Migraine Pathophysiology”, Headache 2018; 58 Suppl 1:4-16; Dodick D W, “Migraine,” Lancet 2018:391:1315-30; Goadsby P J et al., “Pathophysiology of Migraine: a disorder of sensory processing”, Physiol Rev 2017; 97:553-622.

The prodrome is the earliest clinical manifestation of a migraine attack. Prodrome symptoms can be broadly grouped into four categories: cognitive and mood changes (e.g., concentration change, difficulties reading, memory complaints, confusion, disorientation, and mood changes); homeostatic and hormonal changes (e.g., food cravings, thirst, polyuria, yawning, altered sleep-wake cycle, and changes in alertness); migrainous and sensory sensitivities (mild head discomfort, photophobia, phonophobia, osmophobia, neck discomfort, allodynia, and nausea); and cranial autonomic symptoms (lacrimation, nasal stuffiness, rhinorrhea, aural fullness, abnormal taste, and sensation of throat swelling). The prodrome occurs hours to days before the onset of the headache phase. See Headache Classification Committee of the International Headache Society, The International Classification of Headache Disorders, 3^{rd} edition, Cephalalgia 2018; 38:1-211.

Treatment guidelines from the American Headache Society recommend treating acute migraine as early as possible after the onset of headache pain, as delayed treatment may increase pain severity and disability. See Marmura M J1, Silberstein S D, Schwedt T J. The acute treatment of migraine in adults: The American Headache Society Evidence Assessment of Migraine Pharmacotherapies. Headache. 2015 January; 55(1):3-20. Early treatment of a migraine headache may improve rates of pain freedom and pain relief, and reduce headache recurrence and rescue medication use when compared with treatment of a moderate to severe migraine headache. See Dowson A. Can oral 311C90, a novel (5-HT_{1D}) agonist, prevent migraine headache when taken during an aura? Eur Neurol. 1996; 36:28-31. However, treating a migraine at the beginning of the headache phase may still be a suboptimal treatment for patients, as patients must still contend with both headache and nonheadache migraine symptoms.

The possibility of treating a migraine attack with a triptan in the preheadache phases (i.e., prodrome or aura) has been investigated previously with mixed results. It is difficult to draw any definitive conclusions regarding the efficacy of triptans when administered during the prodrome or aura because of significant design limitations with these studies (e.g., small sample sizes, open-label design, and short timeframe from aura to headache).

Notably, prodromal symptoms are prevalent in people with migraine. In a prospective electronic diary study, prodromal symptoms were found in 82% of participants. See Giffin N J, Ruggiero L, Lipton R B, et al. Neurology 2003; 60:935-40. In 72% of these individuals, prodromal symptoms could accurately predict the headache phase. In a clinic sample of 461 patients with migraine who were questioned about the presence of 12 predefined prodromal symptoms, 86.9% reported at least one prodromal symptom. See Schoonman G G, et al. Cephalalgia 2006; 26:1209-13. As disclosed herein, the prodrome phase opens a window for using acute treatment that could prevent or attenuate the headache phase, reduce attack-related disability, and improve patient outcomes.

The present disclosure provides the discovery that treatment of migraine with a CGRP receptor antagonist such as ubrogepant during the initial phase of a migraine attack, before the onset of headache, can prevent the development of a moderate or severe headache. In embodiments, the present disclosure provides a method of treating migraine, the method comprising administering a CGRP receptor antagonist during the prodrome phase of the migraine attack. In embodiments, the CGRP receptor antagonist is selected from the group consisting of ubrogepant, atogepant, rimegepant, and zavegepant, or a pharmaceutically acceptable salt thereof. In embodiments, the CGRP receptor antagonist is ubrogepant. In embodiments, treatment with ubrogepant during the prodrome stage can prevent or attenuate the headache phase, reduce attack-related disability, and improve patient outcomes.

Ubrogepant, the active ingredient of Ubrelvy®, is a calcitonin gene-related peptide (CGRP) receptor antagonist approved for the acute treatment of migraine with or without aura in adults. Ubrogepant has the following structure:

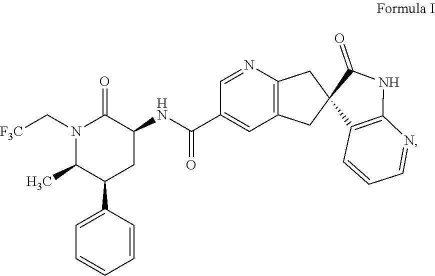 [SMILES: C[C@@H]1[C@H](c2ccccc2)C[C@H](NC(=O)c2cnc3c(c2)C[C@@]2(C3)C(=O)Nc3ncccc32)C(=O)N1CC(F)(F)F]

The chemical name of ubrogepant is (3′S)—N-((3S,5S,6R)-6-methyl-2-oxo-5-phenyl-1-(2,2,2-trifluoroethyl)piperidin-3-yl)-2′-oxo-1′,2′,5,7-tetrahydrospiro[cyclopenta[b]pyridine-6,3′-pyrrolo[2,3-b]pyridine]-3-carboxamide. The molecular formula is C₂₉H₂₆F₃N₅O₃ and the molecular weight is 549.6. Preparation of ubrogepant is described, for example, in U.S. Pat. No. 8,481,556, the entire disclosure of which is incorporated by reference in its entirety.

In embodiments, the present disclosure provides methods for the treatment of migraine, in particular the acute treatment of migraine. In embodiments, the methods of the present disclosure involve administering a CGRP receptor antagonist (e.g., ubrogepant, atogepant, rimegepant, zavegepant) during the prodrome stage of a migraine. In embodiments, the CGRP receptor antagonist is ubrogepant.

For example, in embodiments, the present disclosure provides a method for the treatment of migraine, the method comprising administering ubrogepant or a pharmaceutically acceptable salt thereof to a patient in need of treatment for migraine, wherein ubrogepant is administered during the prodrome stage of a migraine attack. In embodiments, ubrogepant is administered at a dose of 50 mg or 100 mg. In embodiments, ubrogepant is administered at a dose of 50 mg. In embodiments, ubrogepant is administered at a dose of 100 mg. In embodiments, a second dose of ubrogepant may be administered at least two hours after the first dose of ubrogepant. In embodiments, the second dose of ubrogepant is 50 mg or 100 mg.

In embodiments, the present disclosure provides methods for the acute treatment of migraine, the method comprising administering a CGRP receptor antagonist to a patient in need thereof, wherein the patient is experiencing a migraine attack but has not yet developed a migraine headache. In embodiments, the CGRP receptor antagonist is selected from the group consisting of ubrogepant, atogepant, rimegepant, and zavegepant. In embodiments, the CGRP receptor antagonist is an oral CGRP receptor antagonist. In embodiments, the CGRP receptor antagonist is ubrogepant. In embodiments, treatment with a CGRP receptor antagonist when the patient is experiencing a migraine attack but before the patient has developed a migraine headache prevents or reduces the severity of a migraine headache within 24 hours or within 36 hours or within 48 hours of administration. In embodiments, treatment with a CGRP receptor antagonist when the patient is experiencing a migraine attack but before the patient has developed a migraine headache prevents the development of a moderate or severe headache within 24 hours of administration of the CGRP receptor antagonist. In embodiments, treatment with a CGRP receptor antagonist when the patient is experiencing a migraine attack but before the patient has developed a migraine headache prevents the development of a moderate or severe headache within 48 hours of administration of the CGRP receptor antagonist. In embodiments, treatment with a CGRP receptor antagonist when the patient is experiencing a migraine attack but before the patient has developed a migraine headache prevents the development of a headache of any intensity within 24 hours of administration of the CGRP receptor antagonist.

In embodiments, treatment with a CGRP receptor antagonist such as ubrogepant during the prodrome phase of a migraine attack can prevent or reduce the intensity of symptoms associated with migraine. In embodiments, symptoms of a migraine attack may include, for example, pain, aura, photophobia, phonophobia, fatigue, neck pain, and dizziness. In embodiments, the methods of the present disclosure may result in freedom from symptoms associated with migraine. In embodiments, the administration of a CGRP receptor antagonist such as ubrogepant during the prodrome phase of migraine as described in the present disclosure may provide for fewer symptoms of migraine or symptoms of reduced intensity. In embodiments, treatment with a CGRP receptor antagonist such as ubrogepant during the prodrome stage of a migraine attack as provided in the present disclosure may result in the symptoms of a migraine attack being reduced or eliminated. In embodiments, treatment with ubrogepant during the prodrome stage of a migraine attack reduces the severity of or eliminates migraine headache for at least 24 hours or at least 36 hours or at least 48 hours after administration of the CGRP receptor antagonist. In embodiments, treatment with ubrogepant during the prodrome stage of a migraine attack reduces the severity of or eliminates a migraine symptom selected from the group consisting of pain, aura, photophobia, phonophobia, fatigue, neck pain, and dizziness for at least 24 hours or at least 36 hours or at least 48 hours after administration of ubrogepant.

In embodiments, treatment with a CGRP receptor antagonist such as ubrogepant during the initial phase of a migraine attack (i.e., prodrome or premonitory phase) before the onset of headache prevents the development of a migraine headache. In embodiments, treatment during the prodrome stage of a migraine attack prevents the development of a moderate or severe headache within 24 hours of administration of the CGRP receptor antagonist, or within 36 hours, or within 48 hours. In embodiments, treatment with a CGRP receptor antagonist such as ubrogepant during the prodrome/premonitory phase prevents the development of a headache of any intensity within 24 hours of administering ubrogepant.

In embodiments, treatment with ubrogepant during the prodrome stage of a migraine attack results in a statistically significant treatment effect with respect to the absence of a headache of moderate or severe intensity within 24 hours of administering ubrogepant as compared to placebo. In embodiments, treatment with ubrogepant during the prodrome stage of a migraine attack results in a statistically significant treatment effect with respect to the absence of a headache of moderate or severe intensity within 48 hours of administering ubrogepant as compared to placebo. In embodiments, treatment with ubrogepant during the prodrome stage of a migraine attack results in a statistically significant treatment effect with respect to the absence of a headache of any intensity within 24 hours of administering ubrogepant as compared to placebo.

In embodiments, ubrogepant is administered when a patient is experiencing at least one prodrome symptom. In embodiments, the at least one prodrome symptom is selected from the group consisting of cognitive and mood changes (e.g., concentration change, difficulties reading, memory complaints, confusion, disorientation, and mood changes); homeostatic and hormonal changes (e.g., food cravings, thirst, polyuria, yawning, altered sleep-wake cycle, and changes in alertness); migrainous and sensory sensitivities (mild head discomfort, photophobia, phonophobia, osmophobia, neck discomfort, allodynia, and nausea); and cranial autonomic symptoms (lacrimation, nasal stuffiness, rhinorrhea, aural fullness, abnormal taste, and sensation of throat swelling). In embodiments, the at least one prodrome symptom is selected from the group consisting of sensitivity to light; tired/sleepy/fatigue; neck pain/stiff neck; sensitivity to sound; dizziness/lightheadedness/vertigo/imbalance; irritability; nausea; difficulty concentrating; muscle pain/aching; blurred vision; difficulty thinking; yawning; sensitivity to smell; temperature change (e.g., feeling especially cold or warm, chills, sweats); loss of appetite; thirst; emotional; stuffy nose; pale or flushed face; teary eyes, red eyes; difficulty reading or writing; sensitive skin; food craving/hunger; frequent urination; difficulty speaking; changes in bowel movement (e.g., diarrhea, constipation); excessive energy/hyperactivity; and vomiting. In embodiments, ubrogepant is administered when a patient is experiencing at least one prodrome symptom but does not have a migraine headache.

Prodrome symptoms, in addition to the pain of the headache, have a significant impact on the quality of life and functioning of individuals with migraine. In embodiments, treatment with ubrogepant during the prodrome stage of a migraine attack reduces the severity of or eliminates at least one prodrome symptom. In embodiments, the prodrome symptom is selected from the group consisting of sensitivity to light; tired/sleepy/fatigue; neck pain/stiff neck; sensitivity to sound; dizziness/lightheadedness/vertigo/imbalance; irritability; nausea; difficulty concentrating; muscle pain/aching; blurred vision; difficulty thinking; yawning; sensitivity to smell; temperature change (e.g., feeling especially cold or warm, chills, sweats); loss of appetite; thirst; emotional; stuffy nose; pale or flushed face; teary eyes, red eyes; difficulty reading or writing; sensitive skin; food craving/hunger; frequent urination; difficulty speaking; changes in bowel movement (e.g., diarrhea, constipation); excessive energy/hyperactivity; and vomiting.

In embodiments, the present disclosure provides a method of treating migraine, the method comprising administering a CGRP receptor antagonist (e.g., ubrogepant) to a patient in need thereof, wherein the CGRP receptor antagonist is administered during the prodrome stage of the migraine attack, and wherein the patient is experiencing at least one prodrome symptom. In embodiments, ubrogepant is administered at a dose of 50 mg or 100 mg. In embodiments, ubrogepant is administered at a dose of 50 mg. In embodiments, ubrogepant is administered at a dose of 100 mg. In embodiments, treatment with ubrogepant is effective to reduce or eliminate the at least one prodrome symptom. In embodiments, treatment with ubrogepant is further effective to reduce or eliminate migraine headache in the patient.

For example, in embodiments, the present disclosure provides a method of treating migraine, comprising administering a CGRP receptor antagonist (e.g., ubrogepant) to a patient in need thereof, wherein the CGRP receptor antagonist is administered during the prodrome stage, and wherein the patient is experiencing sensitivity to light. In embodiments, treatment with ubrogepant is effective to reduce or eliminate the patient's sensitivity to light. In embodiments, treatment with ubrogepant is effective to reduce or eliminate the patient's sensitivity to light within about 2 hours of administering ubrogepant. In embodiments, treatment with ubrogepant is effective to reduce or eliminate the patient's sensitivity to light within about 3 hours, or within about 4 hours, or within about 6 hours, or within about 8 hours, or within about 24 hours, or within about 48 hours. In embodiments, treatment with ubrogepant is effective to reduce or eliminate the patient's sensitivity to light for at least about 8 hours, or at least about 24 hours, or at least about 48 hours. In embodiments, treatment with ubrogepant is further effective to reduce or eliminate migraine headache in the patient. In embodiments, ubrogepant is administered at a dose of 50 mg or 100 mg. In embodiments, ubrogepant is administered at a dose of 50 mg. In embodiments, ubrogepant is administered at a dose of 100 mg.

In embodiments, the present disclosure provides a method of treating migraine, comprising administering a CGRP receptor antagonist (e.g., ubrogepant) to a patient in need thereof, wherein the CGRP receptor antagonist is administering during the prodrome stage, and wherein the patient is experiencing at least one prodrome symptom, wherein the prodrome symptom is fatigue. In embodiments, treatment with ubrogepant is effective to reduce or eliminate the patient's fatigue within about 3 hours after administering ubrogepant. In embodiments, treatment with ubrogepant is effective to reduce or eliminate the patient's fatigue within about 3 hours, or within about 4 hours, or within about 6 hours, or within about 8 hours, or within about 24 hours, or within about 48 hours. In embodiments, treatment with ubrogepant is effective to reduce or eliminate the patient's fatigue for at least about 8 hours, or at least about 24 hours, or at least about 48 hours. In embodiments, treatment with ubrogepant is further effective to reduce or eliminate migraine headache in the patient. In embodiments, ubrogepant is administered at a dose of 50 mg or 100 mg. In embodiments, ubrogepant is administered at a dose of 50 mg. In embodiments, ubrogepant is administered at a dose of 100 mg.

In embodiments, the present disclosure provides a method of treating migraine, comprising administering a CGRP receptor antagonist (e.g., ubrogepant) to a patient in need thereof, wherein the CGRP receptor antagonist is administering during the prodrome stage, and wherein the patient is experiencing at least one prodrome symptom, wherein the prodrome symptom is neck pain. In embodiments, treatment with ubrogepant is effective to reduce or eliminate the patient's neck pain within about 3 hours after administering ubrogepant. In embodiments, treatment with ubrogepant is effective to reduce or eliminate the patient's neck pain within about 3 hours, or within about 4 hours, or within about 6 hours, or within about 8 hours, or within about 24 hours, or within about 48 hours. In embodiments, treatment with ubrogepant is effective to reduce or eliminate the patient's neck pain for at least about 8 hours, or at least about 24 hours, or at least about 48 hours. In embodiments, treatment with ubrogepant is further effective to reduce or eliminate migraine headache in the patient. In embodiments, ubrogepant is administered at a dose of 50 mg or 100 mg. In embodiments, ubrogepant is administered at a dose of 50 mg. In embodiments, ubrogepant is administered at a dose of 100 mg.

In embodiments, the present disclosure provides a method of treating migraine, comprising administering a CGRP receptor antagonist (e.g., ubrogepant) to a patient in need thereof, wherein the CGRP receptor antagonist is administering during the prodrome stage, and wherein the patient is experiencing at least one prodrome symptom, wherein the prodrome symptom is sensitivity to sound. In embodiments, treatment with ubrogepant is effective to reduce or eliminate the patient's sensitivity to sound within about 1 hour, or within about 2 hours, or within about 3 hours, or within about 4 hours, or within about 6 hours, or within about 8 hours, or within about 24 hours, or within about 48 hours. In embodiments, treatment with ubrogepant is effective to reduce or eliminate the patient's sensitivity to sound for at least about 8 hours, or at least about 24 hours, or at least about 48 hours. In embodiments, treatment with ubrogepant is further effective to reduce or eliminate migraine headache in the patient. In embodiments, ubrogepant is administered at a dose of 50 mg or 100 mg. In embodiments, ubrogepant is administered at a dose of 50 mg. In embodiments, ubrogepant is administered at a dose of 100 mg.

In embodiments, the present disclosure provides a method of treating migraine, comprising administering a CGRP receptor antagonist (e.g., ubrogepant) to a patient in need thereof, wherein the CGRP receptor antagonist is administering during the prodrome stage, and wherein the patient is experiencing at least one prodrome symptom, wherein the prodrome symptom is dizziness. In embodiments, treatment with ubrogepant is effective to reduce or eliminate the patient's dizziness within about 2 hours, or within about 3 hours, or within about 4 hours, or within about 6 hours, or within about 8 hours, or within about 24 hours, or within about 48 hours. In embodiments, treatment with ubrogepant is effective to reduce or eliminate the patient's dizziness for at least about 8 hours, or at least about 24 hours, or at least about 48 hours. In embodiments, treatment with ubrogepant is further effective to reduce or eliminate migraine headache in the patient. In embodiments, ubrogepant is administered at a dose of 50 mg or 100 mg. In embodiments, ubrogepant is administered at a dose of 50 mg. In embodiments, ubrogepant is administered at a dose of 100 mg.

While the biology of the prodrome phase of migraine is still being elucidated, without being bound to any particular theory, hypothalamic activation and functional coupling between the hypothalamus and trigeminal nucleus caudalis has been demonstrated. CGRP receptors are highly expressed in these and other central nervous system regions involved in migraine pathogenesis, as well as in peripheral trigeminovascular nociceptors. Salivary CGRP-like immunoreactivity levels are increased between migraine attacks and progressively increase in the pre-headache and headache phase. These findings suggest a pathophysiological role for CGRP in the prodrome. The presence of a CGRP-receptor antagonist, such as ubrogepant, during the prodrome, may reduce CGRP-mediated activation of hypothalamic and other accessible central nervous system sites involved in the progression of the migraine attack.

The present disclosure may be more clearly understood by reference to the following examples, which are included for purposes of illustration and are not intended to be limiting.

### EXAMPLES

### Example 1

A phase 3, multicenter, randomized, double-blind, placebo-controlled, crossover study was carried out to evaluate the efficacy, safety, and tolerability of oral ubrogepant in the acute treatment of migraine when administered during the prodrome. The study evaluated whether treatment during the initial phase of a migraine attack (i.e., the prodrome or premonitory phase) prior to the onset of headache, can attenuate the severity of the headache phase and reduce disability.

Eligible participants were able to treat two qualifying prodrome events, defined as a migraine attack with prodromal symptoms in which the participant was confident a headache would follow within 1-6 hours. Headache could not be present during the prodrome phase and the participant could not have had a headache or used acute treatment within 48 hours prior to the onset of the qualifying prodrome event.

The primary endpoint was absence of moderate or severe intensity headache within 24 hours post-dose. Secondary endpoints were absence of moderate/severe intensity headache within 48 hours, ability to function normally over 24 hours, and absence of headache of any intensity within 24 hours post-dose.

The trial enrolled adults with 2-8 migraine attacks per month who stated that they could identify migraine attacks with prodromal symptoms reliably followed by headache. Using an e-diary, participants reported qualifying events, defined as a migraine attack with prodromal symptoms in which the participant was confident a headache would follow within 1-6 hours.

A total of 920 participants entered e-diary data, with a mean (median) of 5.2 (5.0) qualifying prodrome events reported per participant. Of 4802 qualifying prodrome events, the most common symptoms were sensitivity to light (57.2%), fatigue (50.1%), neck pain (41.9%), sensitivity to sound (33.9%), and dizziness (27.8%). The majority (95.1%) of qualifying events were followed by a headache within 24 hours; 90.5% were followed by a headache within 6 hours; and 81.5% were followed by a headache within 1-6 hours. For each participant, a mean (median) of 84.4% (100%) of their qualifying prodrome events were followed by a headache within 1-6 hours, with 76.9% of participants identifying prodromal symptoms followed by a headache ≥75% of the time. Participants were thus able to identify migraine attacks in which prodromal symptoms were reliably followed by headache within 1-6 hours during the screening period.

Eligible participants were randomized (1:1) to treatment sequences A or B in this crossover study. Participants randomized to Treatment Sequence A received placebo to treat their first qualifying prodrome event and ubrogepant 100 mg to treat their second qualifying prodrome event. For those randomized to Treatment Sequence B, they received the reverse: ubrogepant 100 mg to treat their first qualifying prodrome event and placebo to treat their second qualifying prodrome event.

The primary efficacy endpoint was the absence of a headache of moderate/severe intensity within 24 hours after taking double-blind study intervention during the prodrome. The secondary efficacy endpoints were the absence of a headache of moderate or severe intensity within 48 hours, ability to function normally over 24 hours, and absence of a headache of any intensity within 24 hours after taking double blind study interventions during the prodrome.

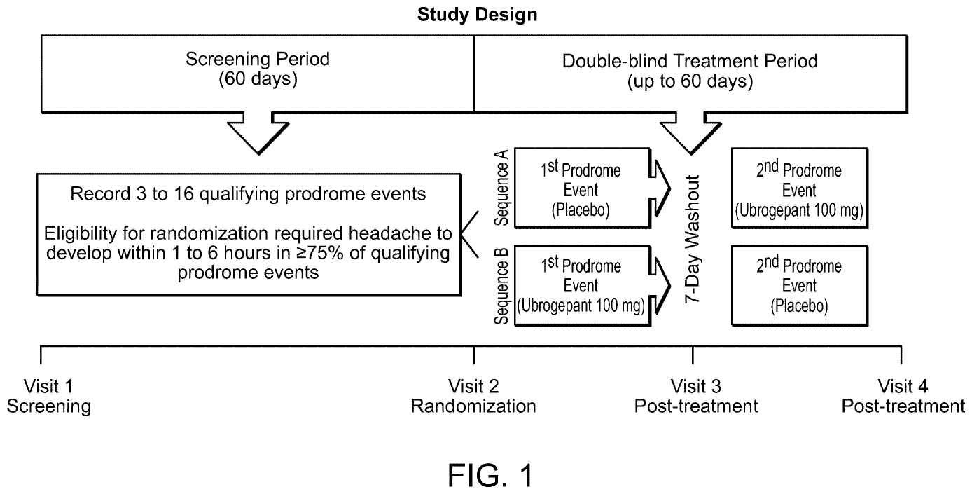

The overall study design is illustrated in FIG. 1. The study included a 60-day screening period during which participants were to demonstrate that they reliably develop headache after experiencing qualifying prodrome events. After meeting eligibility criteria, randomized participants were to treat two qualifying prodrome events with study intervention during the double-blind treatment period (between Visit 2 and Visit 4) (up to 60 days), separated by a clinic visit (visit 3) to ensure a minimum 7-day washout period. A post-treatment visit (visit 4) followed four days after the last treatment was taken.

To be eligible for study participation, participants must be 18 to 75 years of age (inclusive) at Visit 1, have at least a 1-year history of migraine with or without aura consistent with diagnosis according to the ICHD-3 (2018), and experience 2 to 8 migraine attacks with moderate to severe headache per month by history in each of the 3 months prior to screening. At the screening visit, participants were asked whether they could identify prodromal symptoms followed by migraine headache within 1 to 6 hours at least 75% of the time. Participants who responded in the affirmative were asked to demonstrate this by recording all their qualifying prodrome events during the 60-day screening period. After the screening period, to be eligible for randomization, the participant needed to record 3 to 16 qualifying prodrome events with at least 75% of these events followed by a headache, of any intensity, within 1 to 6 hours.

Participants were excluded if they had difficulty distinguishing migraine from tension-type or other headache types, or if they had chronic migraine, a trigeminal autonomic cephalalgia, or a painful cranial neuropathy. Participants who overused medication for migraine or had previous exposure to an injectable anti-CGRP monoclonal antibody within the previous 3 months were also excluded.

A total of 1087 participants were screened and 518 participants were randomized, among them 264 to Treatment Sequence A (placebo/ubrogepant 100 mg) and 254 to Treatment Sequence B (ubrogepant 100 mg/placebo). The majority of participants (438, 84.6%) completed the double-blind period. The discontinuation rate for placebo/ubrogepant 100 mg and ubrogepant 100 mg/placebo were 15.5% and 15.4%, respectively. The main reasons for discontinuation were lack of two qualifying prodrome events: 9.5% and 10.6% for placebo/ubrogepant 100 mg and ubrogepant 100 mg/placebo sequences, respectively. Numbers of participants in analysis populations are shown in Table 1.

TABLE 1
Number of Participants in Analysis Populations
By Treatment Sequence

| | Placebo/Ubrogepant | Ubrogepant | |
|---|---|---|---|
| | 100 mg | 100 mg/Placebo | Total |
| Randomized | 264 | 254 | 518 |
| Safety Population | 247 | 233 | 480 |

By Treatment

| | | Ubrogepant | |
|---|---|---|---|
| | Placebo | 100 mg | Total |
| Randomized | 518 | 518 | 518 |
| Safety Population | 462 | 456 | 480 |
| Modified Intent-to-Treat Population | 449 | 448 | 477 |

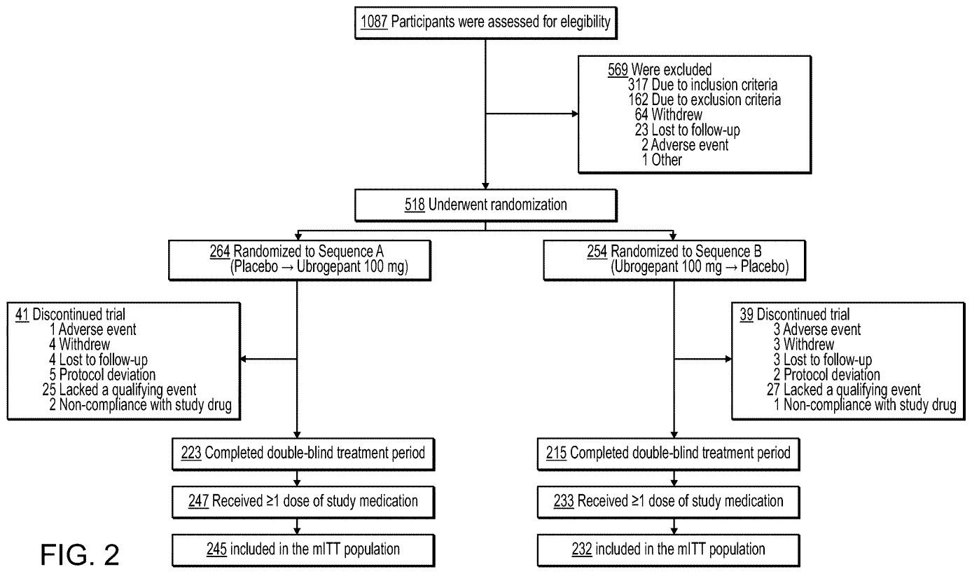

Participant flow is illustrated in FIG. 2. The safety population included all treated participants who took at least one administration of study drug. The modified intent-to-treat (mITT) population included all randomized participants with at least one assessment of headache occurrence within 24 hours after taking double-blind study drug for at least one qualifying prodrome event. The most common reasons for failure to meet inclusion criteria were: lack of 3 to 16 recorded qualifying prodrome events during screening (144 of 317), failure to report headache of any intensity within 1 to 6 hours following at least 75% of qualifying prodrome events (123 of 317), and participant provided a negative response for the ability (by history) to identify migraine attacks in which prodromal symptoms were present and reliably (at least 75% of the time) followed by headache within 1 to 6 hours (23 of 317

Demographic data for the safety population are summarized in Table 2. Overall the mean age was 42.3 years, 87.7% of the participants were female, 88.1% of the participants were white and 7.7% were black or African American. The demographics were balanced between treatment sequences.

TABLE 2
Demographics - Safety Population

| | Treatment Sequence | | | | | |
|---|---|---|---|---|---|---|
| | Placebo/ | | Ubrogepant | | | |
| | Ubrogepant | | 100 mg/ | | | |
| | 100 mg | | Placebo | | Total | |
| | (N = 247) | | (N = 233) | | (N = 480) | |
| Age (years) | | | | | | |
| Mean (SD) | 41.7 | (12.63) | 42.9 | (13.10) | 42.3 | (12.86) |
| Median | 41.0 | | 42.0 | | 42.0 | |
| Min, Max | 18, 74 | | 19, 71 | | 18, 74 | |
| n | 247 | | 233 | | 480 | |
| Sex, n (%) | | | | | | |
| Male | 31 | (12.6) | 28 | (12.0) | 59 | (12.3) |
| Female | 216 | (87.4) | 205 | (88.0) | 421 | (87.7) |
| Race, n (%) | | | | | | |
| White | 214 | (86.6) | 209 | (89.7) | 423 | (88.1) |
| Black or African | 22 | (8.9) | 15 | (6.4) | 37 | (7.7) |
| American | | | | | | |
| Asian | 7 | (2.8) | 4 | (1.7) | 11 | (2.3) |
| American Indian or | 0 | (0.0) | 0 | (0.0) | 0 | (0.0) |
| Alaska Native | | | | | | |
| Native Hawaiian or | 1 | (0.4) | 1 | (0.4) | 2 | (0.4) |
| Other Pacific Islander | | | | | | |
| Multiple | 2 | (0.8) | 4 | (1.7) | 6 | (1.3) |
| Missing | 1 | (0.4) | 0 | (0.0) | 1 | (0.2) |
| Ethnicity, n (%) | | | | | | |
| Hispanic or Latino | 17 | (6.9) | 15 | (6.4) | 32 | (6.7) |
| Not Hispanic or Latino | 229 | (92.7) | 216 | (92.7) | 445 | (92.7) |
| Missing | 1 | (0.4) | 2 | (0.9) | 3 | (0.6) |

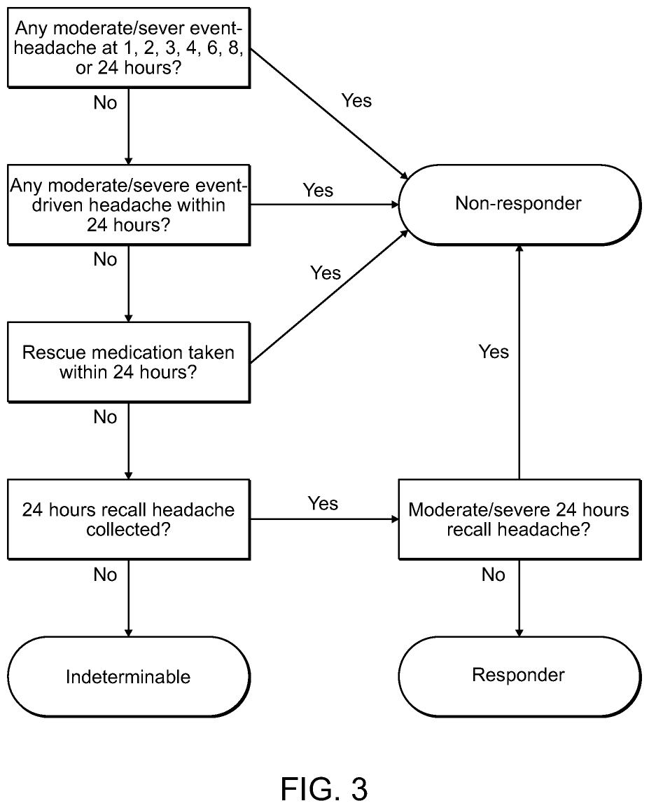

The primary efficacy endpoint was the absence of a headache of moderate/severe intensity within 24 hours after taking double-blind study intervention during the prodrome. The status of absence of a headache of moderate/severe intensity within 24 hours was decided based on all observed data including headache reported at a series of scheduled timepoints, reported event-driven assessments (i.e., onset of headache between scheduled timepoints), 24-hour recall of headache, and whether a patient took rescue medication. To be classified as a treatment responder, a participant was required to not report a headache of moderate/severe intensity within 24 hours and report no use of rescue medication within 24 hours as outlined in FIG. 3. Participants who reported a headache or use of acute medication at any of these assessments were considered nonresponders to treatment. Participants who reported no headache occurrence at the pre-specified time points were required to answer an additional recall question affirming they did not experience a headache of moderate/severe intensity in the last 24 hours. Any participant who answered all questions at prespecified timepoints but failed to answer this 24-hour recall question was considered indeterminable.

Table 3 presents the analysis results for the primary efficacy endpoint in the mITT Population, which demonstrated statistically significant treatment differences between ubrogepant and placebo on the absence of a headache of moderate/severe intensity within 24 hours. The sensitivity analyses for testing carryover effect, period 1 data only, and non-responder imputation analyses are also shown.

TABLE 3
Summary of Primary Efficacy Endpoint of Absence
of a Headache of Moderate/Severe Intensity within
24 Hours - Modified Intent-to-Treat Population

| | |
|---|---|
| Placebo | Ubrogepant 100 mg |

Primary Efficacy Endpoint: Absence of a headache of
moderate/severe intensity within 24 hours
Primary Analysis: Observed Cases

| N1 | 423 | | 418 | |
|---|---|---|---|---|
| Responder, n (%) | 121 | (28.6) | 190 | (45.5) |
| Nonresponder, n (%) | 302 | (71.4) | 228 | (54.5) |
| Odds Ratio (95% CI) vs. Placebo | | | 2.09 | (1.63, 2.69) |

| | |
|---|---|
| P-value vs. Placebo [1] | <.0001 |

Sensitivity Analysis: Testing carryover effect

| N1 | 423 | | 418 | |
|---|---|---|---|---|
| Responder, n (%) | 121 | (28.6) | 190 | (45.5) |
| Nonresponder, n (%) | 302 | (71.4) | 228 | (54.5) |
| Odds Ratio (95% CI) vs. Placebo | | | 2.09 | (1.63, 2.69) |

| | |
|---|---|
| Carryover effect P-value [2] | 0.9305 |

Sensitivity Analysis: Period 1 data only

| N1 | 224 | | 209 | |
|---|---|---|---|---|
| Responder, n (%) | 67 | (29.9) | 99 | (47.4) |
| Nonresponder, n (%) | 157 | (70.1) | 110 | (52.6) |
| Odds Ratio (95% CI) vs. Placebo | | | 2.11 | (1.42, 3.13) |

| | |
|---|---|
| P-value vs. Placebo [3] | 0.0002 |

Sensitivity Analysis: Non-Responder Imputation

| N1 | 449 | | 448 | |
|---|---|---|---|---|
| Responder, n (%) | 121 | (26.9) | 190 | (42.4) |
| Nonresponder, n (%) | 328 | (73.1) | 258 | (57.6) |
| Odds Ratio (95% CI) vs. Placebo | | | 2.01 | (1.58, 2.57) |

| | |
|---|---|
| P-value vs. Placebo [1] | <.0001 |

N1 = Number of participants whose absence/presence of moderate/severe intensity headache within 24 hours can be determined or available.
Non-Responder Imputation for Indeterminable: Indeterminable primary endpoints are set as non-responder.
[1] Odds ratio (95% CI) and p-value are based on generalized linear mixed model (GLMM) with treatment group and treatment period as categorical fixed effects. An unstructured covariance matrix is selected for the covariance matrix of the residual effect for the repeated measurements (corresponding to the 2 qualifying prodrome events) within a participant.
[2] Odds ratio (95% CI) and p-value are based on generalized linear mixed model (GLMM) with treatment group, treatment period, and period by treatment as interaction effect (to examine carryover effect) as categorical fixed effects. An unstructured covariance matrix is selected for the covariance matrix of the residual effect for the repeated measurements (corresponding to the 2 qualifying prodrome events) within a participant.
[3] Odds ratio (95% CI) and p-value are based on logistic regression model with treatment group as explanatory variable.

Treatment with ubrogepant 100 mg resulted in a statistically significant higher response rate compared to treatment with placebo for the primary efficacy endpoint, absence of a headache of moderate/severe intensity within 24 hours after taking double-blind study intervention during the prodrome. After taking double-blind ubrogepant 100 mg during the prodrome, 45.5% of participants reported absence of a headache of moderate/severe intensity within 24 hours compared with 28.6% of participants after taking placebo (OR=2.09; p<0.0001 after multiplicity adjustment).

Given the crossover study design, a carryover effect from the first treated qualifying prodrome event to the second treated qualifying prodrome event was tested and, if significant, only Period 1 data were to be used for the primary efficacy analysis. The carryover effect P-value of 0.9305 indicates that there was no carryover effect from the DB treatment of the first event to the DB treatment of the second event. Therefore, the primary analysis model incorporating data from both periods was valid. Analysis of Period 1 data only was performed as an additional sensitivity analysis. The additional sensitivity analyses showed statistically significant higher response rates for ubrogepant 100 mg compared with placebo for the primary efficacy endpoint, and therefore are supportive of and confirm the robustness of the primary analysis of the primary efficacy endpoint.

Secondary endpoints included the absence of headache of moderate/severe intensity within 48 hours (secondary endpoint 1), ability to function normally over 24 hours (secondary endpoint 2), and absence of headache of any intensity within 24 hours (secondary endpoint 3) after taking study drug.

Table 4 presents the analysis results for the absence of a headache of moderate/severe intensity within 48 hours in the mITT population, which demonstrated statistically significant treatment difference between ubrogepant and placebo on the absence of a headache of moderate/severe intensity within 48 hours. After taking double-blind ubrogepant 100 mg during the prodrome, 40.7% of participants reported absence of headache of moderate/severe intensity within 48 hours compared with 24.6% of participants after taking placebo (OR-2.13; p<0.0001 after multiplicity adjustment). This difference between treatments is similar to the difference seen within 24 hours after dosing.

TABLE 4
Summary of Secondary Efficacy Endpoint - Absence
of a Headache of Moderate/Severe Intensity within
48 Hours - Modified Intent to Treat population

| | |
|---|---|
| Placebo | Ubrogepant 100 mg |

Secondary Efficacy Endpoint: Absence of a
Headache of Moderate/Severe Intensity within 48 Hour

| N1 | 407 | 391 | |
|---|---|---|---|
| Responder, n (%) | 100 (24.6) | 159 | (40.7) |
| Nonresponder, n (%) | 307 (75.4) | 232 | (59.3) |
| Odds Ratio vs. Placebo (95% CI) | | 2.13 | (1.63, 2.78) |

| | |
|---|---|
| P-value vs. Placebo [1] | <.0001 |

N1 = Number of participants whose absence/presence of moderate/severe intensity headache within 48 hours can be determined.
[1] Odds ratio (95% CI) and p-value are based on generalized linear mixed model (GLMM) with treatment group and treatment period as categorical fixed effects. An unstructured covariance matrix is selected for the covariance matrix of the residual effect for the repeated measurements (corresponding to the 2 qualifying prodrome events) within a participant.

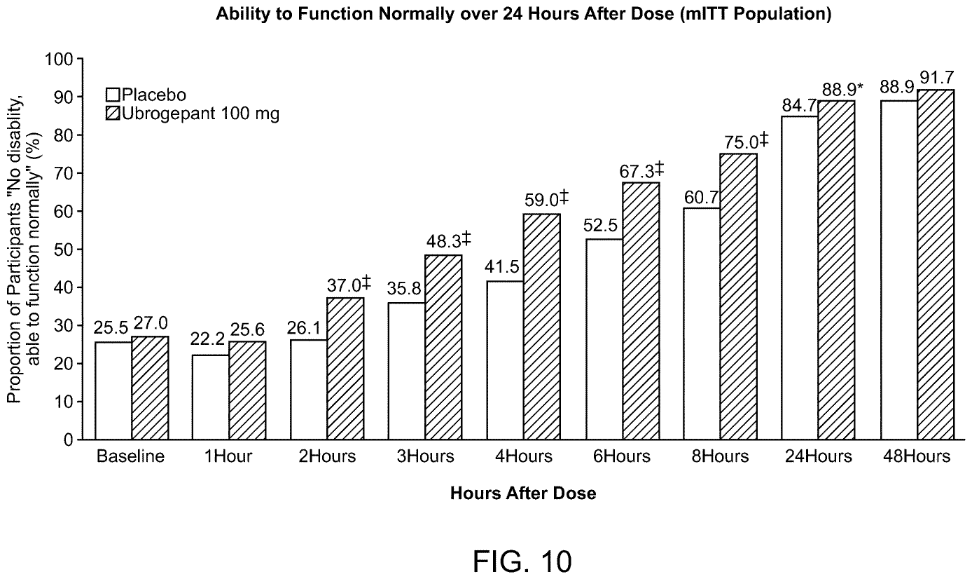

Table 5 and FIG. 10 present the analysis results for ability to function normally over 24 hours in the mITT population, which demonstrated statistically significant treatment differences between ubrogepant and placebo on ability to function normally over 24 hours. A responder for ability to function normally is a participant having a score of 0 (no disability, able to function normally) on the Functional Disability Scale (FDS), and a non-responder is one having a score of 1 (Mildly impaired, can still do everything but with difficulty) to 3 (Severely impaired, cannot do all or most things, bed rest may be necessary).

TABLE 5
Summary of Secondary Efficacy Endpoint - Ability
to Function Normally Over 24 Hours Post
Dose - Modified Intent to Treat Population

| | |
|---|---|
| Placebo | Ubrogepant 100 mg |

Secondary Efficacy Endpoint: Ability to Function Normally Over
24 Hours: the primary comparison is the geometric mean odds ratio
for ubrogepant vs. placebo over all timepoints through 24 hours

| Baseline, n/N1 (%) | 113/449 | (25.2) | 121/448 | (27.0) |
|---|---|---|---|---|
| 1 hour | | | | |
| N1 | 432 | | 418 | |
| Responder, n (%) | 96 | (22.2) | 107 | (25.6) |
| Nonresponder, n (%) | 336 | (77.8) | 311 | (74.4) |
| Odds Ratio vs. | | | 1.29 | (0.92, 1.80 |
| Placebo (95% CI) | | | | |
| P-value vs. Placebo | | | 0.1453 | |
| 2 hours | | | | |
| N1 | 421 | | 422 | |
| Responder, n (%) | 110 | (26.1) | 156 | (37.0) |
| Nonresponder, n (%) | 311 | (73.9) | 266 | (63.0) |
| Odds Ratio vs. | | | 1.73 | (1.30, 2.30) |
| Placebo (95% CI) | | | | |
| P-value vs. Placebo | | | 0.0002 | |
| 3 hours | | | | |
| N1 | 419 | | 412 | |
| Responder, n (%) | 150 | (35.8) | 199 | (48.3) |
| Nonresponder, n (%) | 269 | (64.2) | 213 | (51.7) |
| Odds Ratio vs. | | | 1.71 | (1.32, 2.22) |
| Placebo (95% CI) | | | | |
| P-value vs. Placebo | | | <.0001 | |
| 4 hours | | | | |
| N1 | 417 | | 415 | |
| Responder, n (%) | 173 | (41.5) | 245 | (59.0) |
| Nonresponder, n (%) | 244 | (58.5) | 170 | (41.0) |
| Odds Ratio vs. | | | 1.97 | (1.54, 2.52) |
| Placebo (95% CI) | | | | |
| P-value vs. Placebo | | | <.0001 | |
| 6 hours | | | | |
| N1 | 400 | | 395 | |
| Responder, n (%) | 210 | (52.5) | 266 | (67.3) |
| Nonresponder, n (%) | 190 | (47.5) | 129 | (32.7) |
| Odds Ratio vs. | | | 1.78 | (1.40, 2.27) |
| Placebo (95% CI) | | | | |
| P-value vs. Placebo | | | <.0001 | |
| 8 hours | | | | |
| N1 | 394 | | 388 | |
| Responder, n (%) | 239 | (60.7) | 291 | (75.0) |
| Nonresponder, n (%) | 155 | (39.3) | 97 | (25.0) |
| Odds Ratio vs. | | | 1.84 | (1.41, 2.38) |
| Placebo (95% CI) | | | | |
| P-value vs. Placebo | | | <.0001 | |
| 24 hours | | | | |
| N1 | 404 | | 413 | |
| Responder, n (%) | 342 | (84.7) | 367 | (88.9) |
| Nonresponder, n (%) | 62 | (15.3) | 46 | (11.1) |
| Odds Ratio vs. | | | 1.41 | (0.99, 2.00) |
| Placebo (95% CI) | | | | |
| P-value vs. Placebo | | | 0.0558 | |
| Overall | | | | |
| Geometric Mean Odds Ratio | | | 1.66 | (1.40, 1.96) |
| vs. Placebo (95% CI) | | | | |

| | |
|---|---|
| P-value vs. Placebo | <.0001 |

N1 = Number of participants with non-missing ability to function normally assessment at each timepoint after dose.
Geometric mean of the odds ratios (95%) and p-value are based on generalized estimating equation (GEE) model using observed data at 1, 2, 3, 4, 6, 8, and 24 hours after dose. The model includes period, treatment, post dose scheduled timepoint, treatment-by-time interaction as categorical fixed effects, predose baseline FDS score for the period, and predose baseline-by-time interaction as covariates with logit link and unstructured working correlation matrix for the repeated measures within each participant.

These data show that the baseline functional disability scores demonstrate disability associated with untreated prodrome. Treatment with ubrogepant during the prodrome may reduce the disability and functional limitations of the prodrome itself.

Ubrogepant 100 mg administered during the prodrome stage was associated with significantly greater ability to function normally, greater satisfaction with study medication, and improvements in activity limitation compared with placebo. Following treatment of a qualifying prodrome event, a statistically significant higher response for the ability to function normally over 24 hours (secondary endpoint) was observed for ubrogepant 100 mg compared with placebo (OR=1.66; P<0.0001). As early as two hours post-dose, a significantly greater proportion of participants treated with ubrogepant (37.0%) reported the ability to function normally compared with placebo (26.1%) (nominal P=0.0001). As shown in Table 6, a greater proportion of participants who reported a qualifying prodrome event treated with ubrogepant (65.4%) compared with placebo (47.8%) reported little to no activity limitation over 24 hours post-dose (i.e., reported that they were “not at all limited—I could do everything” or “a little limited”) (nominal P<0.0001).

TABLE 6
Responder Rate at 24 hours - Activity Limitation

| | Placebo | Ubrogepant 100 mg | |
|---|---|---|---|
| Responder rate at 24 hours | (n = 449) | (n = 448) | |
| Responders, n/N1 (%) | 193/404 (47.8) | 270/413 | (65.4) |
| Odds ratio vs placebo (95% CI) | | 2.07 | (1.61, 2.67) |

| | |
|---|---|
| P-value vs placebo | <0.0001 |

N1, Number of participants with non-missing activity limitation assessment at the timepoint after dose. Responders were those who recorded “not at all limited - I could do everything” or “a little limited”

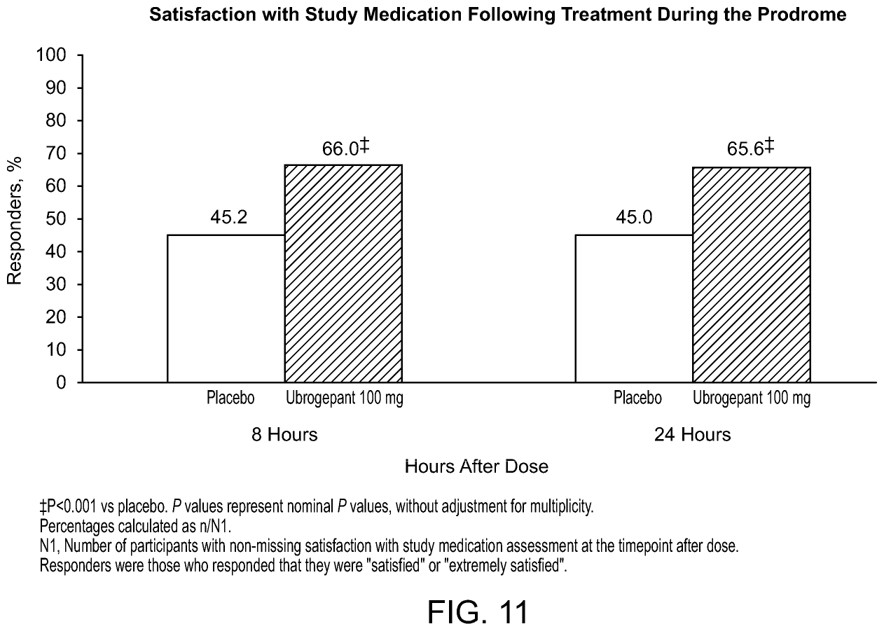

As shown in Table 7 and FIG. 11, at least 24 hours post-dose, a greater proportion of participants treated with ubrogepant (65.6%) reported being “satisfied” or “extremely satisfied” with treatment compared with placebo (45.0%; nominal P<0.0001). Similar responder rates were seen at 24 hours post-dose (nominal P<0.0001).

TABLE 7
Satisfaction with Study Medication Following
Treatment During the Prodrome

| | Placebo | Ubrogepant 100 mg | |
|---|---|---|---|
| Responder rate over time | (n = 449) | (n = 448) | |
| 8 Hours, n/N1 (%) | 177/392 (45.2) | 256/388 | (66.0) |
| Odds ratio vs placebo (95% CI) | | 2.37 | (1.78, 3.15) |
| P-value vs placebo | | <0.0001 | |
| 24 Hours, n/N1 (%) | 182/404 (45.0) | 271/413 | (65.6) |
| Odds ratio vs placebo (95% CI) | | 2.32 | (1.78, 3.02) |

| | |
|---|---|
| P-value vs placebo | <0.0001 |

P values represent nominal P values, without adjustment for multiplicity.
N1, Number of participants with non-missing satisfaction with study medication assessment at the timepoint after dose.
Responders were those who responded that they were “satisfied” or “extremely satisfied”.

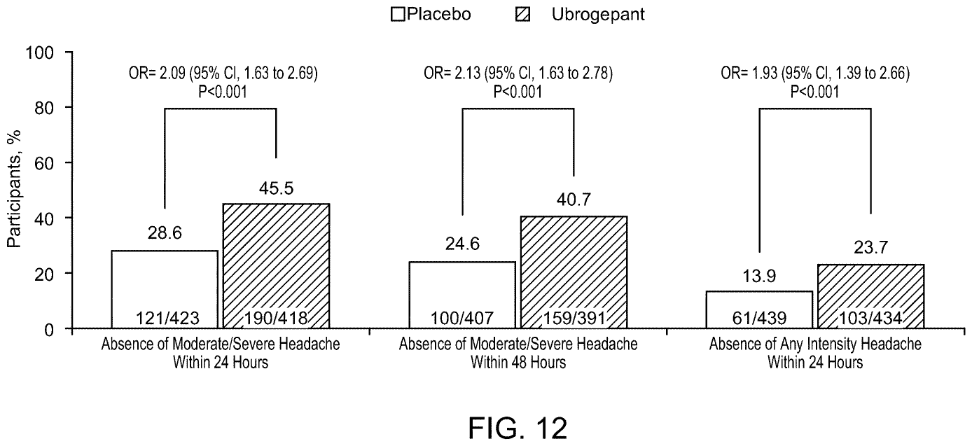

Table 8 presents the analysis results for the absence of a headache of any intensity within 24 hours in the mITT Population, which demonstrated statistically significant treatment differences between ubrogepant and placebo on the absence of a headache of any intensity within 24 hours. Responders are those who did not develop any headache within 24 hours after treatment. FIG. 12 provides a summary of data of Tables 3, 4, and 8. In particular, FIG. 12 shows proportion of participants with absence of moderate/severe headache within 24 hours and 48 hours and absence of any intensity headache within 24 hours after treatment during the prodrome. Odds ratio (OR) (95% confidence interval [CI]) and P value are based on generalized linear mixed model with treatment group and treatment period as categorical fixed effects. Overall, 23.7% of participants reported an absence of a headache of any intensity within 24 hours after taking double-blind ubrogepant 100 mg during the prodrome, compared to 13.9% of participants after taking placebo (OR=1.93; p<0.0001 after multiplicity adjustment).

TABLE 8
Summary of Secondary Efficacy Endpoint - Absence
of a Headache of Any Intensity within 24
Hours - Modified Intent-to-Treat Population

| | |
|---|---|
| Placebo | Ubrogepant 100 mg |

Secondary Efficacy Endpoint: Absence of a
Headache of Any Intensity within 24 Hour

| N1 | 439 | | 434 | |
|---|---|---|---|---|
| Responder, n (%) | 61 | (13.9) | 103 | (23.7) |
| Nonresponder, n (%) | 378 | (86.1) | 331 | (76.3) |

| | | |
|---|---|---|
| Odds Ratio vs. Placebo (95% CI) | 1.93 | (1.39, 2.66) |

| | |
|---|---|
| P-value vs. Placebo [1] | <.0001 |

N1 = Number of participants whose absence/presence of any intensity headache within 24 hours can be determined.
[1] Odds ratio (95% CI) and p-value are based on generalized linear mixed model (GLMM) with treatment group and treatment period as categorical fixed effects. An unstructured covariance matrix is selected for the covariance matrix of the residual effect for the repeated measurements (corresponding to the 2 qualifying prodrome events) within a participant.

Analysis results for the proportion of participants with absence of a headache of any intensity, moderate/severe intensity, and severe intensity are tabulated by time point (within 1 to 48 hours after the dose of study intervention) in Tables 9, 10, and 11. After administration of study intervention during the prodrome, higher response rates for ubrogepant 100 mg compared with placebo were observed starting from 2 hours and extending through 48 hours for the absence of headache of any intensity; from 1 hour and extending through 48 hours for the absence of a headache of moderate/severe intensity; and from 4 hours and extending through to 48 hours for the absence of a headache of severe intensity.

TABLE 9
Absence of a Headache of Any Intensity By Timepoint
(Observed Cases) - mITT Population

| | Placebo | | Ubrogepant 100 mg | |
|---|---|---|---|---|
| Within Hours Post Dose Statistics | (N = 449) | | (N = 448) | |
| 1 Hour | | | | |
| N1 | 436 | | 434 | |
| Responder, n (%) | 230 | (52.8) | 248 | (57.1) |
| Nonresponder, n (%) | 206 | (47.2) | 186 | (42.9) |
| vs. Placebo | | | | |
| Odds Ratio (95%) CI) | | | 1.19 | (0.99, 1.44) |
| P-value | | | 0.0608 | |
| 2 Hours | | | | |
| N1 | 433 | | 438 | |
| Responder, n (%) | 161 | (37.2) | 194 | (44.3) |
| Nonresponder, n (%) | 272 | (62.8) | 244 | (55.7) |
| vs. Placebo | | | | |
| Odds Ratio (95%) CI) | | | 1.34 | (1.08, 1.66) |
| P-value | | | 0.0081 | |
| 3 Hours | | | | |
| N1 | 434 | | 437 | |
| Responder, n (%) | 118 | (27.2) | 164 | (37.5) |
| Nonresponder, n (%) | 316 | (72.8) | 273 | (62.5) |
| vs. Placebo | | | | |
| Odds Ratio (95%) CI) | | | 1.60 | (1.27, 2.01) |
| P-value | | | <.0001 | |
| 4 Hours | | | | |
| N1 | 437 | | 437 | |
| Responder, n (%) | 94 | (21.5) | 143 | (32.7) |
| Nonresponder, n (%) | 343 | (78.5) | 294 | (67.3) |
| vs. Placebo | | | | |
| Odds Ratio (95%) CI) | | | 1.76 (1.35, 2.30) | |
| P-value | | | <.0001 | |
| 6 Hours | | | | |
| N1 | 438 | | 438 | |
| Responder, n (%) | 81 | (18.5) | 127 | (29.0) |
| Nonresponder, n (%) | 357 | (81.5) | 311 | (71.0) |
| vs. Placebo | | | | |
| Odds Ratio (95%) CI) | | | 1.79 | (1.34, 2.40) |
| P-value | | | 0.0001 | |
| 8 Hours | | | | |
| N1 | 441 | | 440 | |
| Responder, n (%) | 75 | (17.0) | 121 | (27.5) |
| Nonresponder, n (%) | 366 | (83.0) | 319 | (72.5) |
| vs. Placebo | | | | |
| Odds Ratio (95%) CI) | | | 1.85 | (1.36, 2.51) |
| P-value | | | <.0001 | |
| 24 Hours | | | | |
| N1 | 439 | | 434 | |
| Responder, n (%) | 61 | (13.9) | 103 | (23.7) |
| Nonresponder, n (%) | 378 | (86.1) | 331 | (76.3) |
| vs. Placebo | | | | |
| Odds Ratio (95%) CI) | | | 1.93 | (1.39, 2.66) |
| P-value | | | <.0001 | |
| 48 Hours | | | | |
| N1 | 432 | | 419 | |
| Responder, n (%) | 47 | (10.9) | 78 | (18.6) |
| Nonresponder, n (%) | 385 | (89.1) | 341 | (81.4) |

| vs. Placebo | | |
|---|---|---|
| Odds Ratio (95%) CI) | 1.90 | (1.33, 2.70) |

| | |
|---|---|
| P-value | 0.0004 |

CI = confidence interval;
N1 = number of participants whose absence/presence of any intensity headache by the timepoint can be determined;
Percentages calculated as 100 × (n/N1)

TABLE 10
Absence of a Headache of Moderate/Severe Intensity
by Timepoint (Observed Cases) - mITT Population

| | Placebo | | Ubrogepant 100 mg | |
|---|---|---|---|---|
| Within Hours Post Dose Statistics | (N = 449) | | (N = 448) | |
| 1 Hour | | | | |
| N1 | 436 | | 433 | |
| Responder, n (%) | 315 | (72.2) | 334 | (77.1) |
| Nonresponder, n (%) | 121 | (27.8) | 99 | (22.9) |
| vs. Placebo | | | | |
| Odds Ratio (95%) CI) | | | 1.30 | (1.03, 1.64) |
| P-value | | | 0.0256 | |
| 2 Hours | | | | |
| N1 | 431 | | 435 | |
| Responder, n (%) | 252 | (58.5) | 281 | (64.6) |
| Nonresponder, n (%) | 179 | (41.5) | 154 | (35.4) |
| vs. Placebo | | | | |
| Odds Ratio (95%) CI) | | | 1.31 | (1.05, 1.64) |
| P-value | | | 0.0156 | |
| 3 Hours | | | | |
| N1 | 427 | | 436 | |
| Responder, n (%) | 201 | (47.1) | 262 | (60.1) |
| Nonresponder, n (%) | 226 | (52.9) | 174 | (39.9) |
| vs. Placebo | | | | |
| Odds Ratio (95%) CI) | | | 1.71 | (1.37, 2.14) |
| P-value | | | <.0001 | |
| 4 Hours | | | | |
| N1 | 432 | | 439 | |
| Responder, n (%) | 185 | (42.8) | 257 | (58.5) |
| Nonresponder, n (%) | 247 | (57.2) | 182 | (41.5) |
| vs. Placebo | | | | |
| Odds Ratio (95%) CI) | | | 1.91 | (1.53, 2.39) |
| P-value | | | <.0001 | |
| 6 Hours | | | | |
| N1 | 433 | | 438 | |
| Responder, n (%) | 166 | (38.3) | 234 | (53.4) |
| Nonresponder, n (%) | 267 | (61.7) | 204 | (46.6) |
| vs. Placebo | | | | |
| Odds Ratio (95%) CI) | | | 1.86 | (1.47, 2.35) |
| P-value | | | <.0001 | |
| 8 Hours | | | | |
| N1 | 433 | | 438 | |
| Responder, n (%) | 151 | (34.9) | 227 | (51.8) |
| Nonresponder, n (%) | 282 | (65.1) | 211 | (48.2) |
| vs. Placebo | | | | |
| Odds Ratio (95%) CI) | | | 2.01 | (1.58, 2.55) |
| P-value | | | <.0001 | |
| 24 Hours | | | | |
| N1 | 423 | | 418 | |
| Responder, n (%) | 121 | (28.6) | 190 | (45.5) |
| Nonresponder, n (%) | 302 | (71.4) | 228 | (54.5) |
| vs. Placebo | | | | |
| Odds Ratio (95%) CI) | | | 2.09 | (1.63, 2.69) |
| P-value | | | <.0001 | |
| 48 Hours | | | | |
| N1 | 407 | | 391 | |
| Responder, n (%) | 100 | (24.6) | 159 | (40.7) |
| Nonresponder, n (%) | 307 | (75.4) | 232 | (59.3) |

| vs. Placebo | | |
|---|---|---|
| Odds Ratio (95%) CI) | 2.13 | (1.63, 2.78) |

| | |
|---|---|
| P-value | <.0001 |

CI = confidence interval;
N1 = number of participants whose absence/presence of moderate/severe headache by the timepoint can be determined;
Percentages calculated as 100 × (n/N1)

TABLE 11
Absence of a Headache of Severe Intensity by
Timepoint (Observed Cases) - mITT Population

| | Placebo | | Ubrogepant 100 mg | |
|---|---|---|---|---|
| Within Hours Post Dose Statistics | (N = 449) | | (N = 448) | |
| 1 Hour | | | | |
| N1 | 432 | | 430 | |
| Responder, n (%) | 412 | (95.4) | 413 | (96.0) |
| Nonresponder, n (%) | 20 | (4.6) | 17 | (4.0) |
| vs. Placebo | | | | |
| Odds Ratio (95%) CI) | | | 1.21 | (0.70, 2.09) |
| P-value | | | 0.4904 | |
| 2 Hours | | | | |
| N1 | 422 | | 419 | |
| Responder, n (%) | 381 | (90.3) | 388 | (92.6) |
| Nonresponder, n (%) | 41 | (9.7) | 31 | (7.4) |
| vs. Placebo | | | | |
| Odds Ratio (95%) CI) | | | 1.33 | (0.88, 2.00) |
| P-value | | | 0.1765 | |
| 3 Hours | | | | |
| N1 | 411 | | 414 | |
| Responder, n (%) | 358 | (87.1) | 375 | (90.6) |
| Nonresponder, n (%) | 53 | (12.9) | 39 | (9.4) |
| vs. Placebo | | | | |
| Odds Ratio (95%) CI) | | | 1.41 | (0.98, 2.03) |
| P-value | | | 0.0652 | |
| 4 Hours | | | | |
| N1 | 404 | | 410 | |
| Responder, n (%) | 335 | (82.9) | 367 | (89.5) |
| Nonresponder, n (%) | 69 | (17.1) | 43 | (10.5) |
| vs. Placebo | | | | |
| Odds Ratio (95%) CI) | | | 1.70 | (1.22, 2.37) |
| P-value | | | 0.0017 | |
| 6 Hours | | | | |
| N1 | 399 | | 401 | |
| Responder, n (%) | 317 | (79.4) | 351 | (87.5) |
| Nonresponder, n (%) | 82 | (20.6) | 50 | (12.5) |
| vs. Placebo | | | | |
| Odds Ratio (95%) CI) | | | 1.77 | (1.30, 2.42) |
| P-value | | | 0.0003 | |
| 8 Hours | | | | |
| N1 | 389 | | 397 | |
| Responder, n (%) | 300 | (77.1) | 345 | (86.9) |
| Nonresponder, n (%) | 89 | (22.9) | 52 | (13.1) |
| vs. Placebo | | | | |
| Odds Ratio (95%) CI) | | | 1.89 | (1.41, 2.53) |
| P-value | | | <.0001 | |
| 24 Hours | | | | |
| N1 | 371 | | 372 | |
| Responder, n (%) | 279 | (75.2) | 318 | (85.5) |
| Nonresponder, n (%) | 92 | (24.8) | 54 | (14.5) |
| vs. Placebo | | | | |
| Odds Ratio (95%) CI) | | | 1.83 | (1.37, 2.45) |
| P-value | | | <.0001 | |
| 48 Hours | | | | |
| N1 | 339 | | 322 | |
| Responder, n (%) | 245 | (72.3) | 266 | (82.6) |
| Nonresponder, n (%) | 94 | (27.7) | 56 | (17.4) |

| vs. Placebo | | |
|---|---|---|
| Odds Ratio (95%) CI) | 1.71 | (1.28, 2.28) |

| | |
|---|---|
| P-value | 0.0003 |

CI = confidence interval;
N1 = number of participants whose absence/presence of severe headache by the timepoint can be determined;
Percentages calculated as 100 × (n/N1)

During the double-blind treatment period, the most common prodromal symptoms reported at baseline, prior to study drug administration, were (ubrogepant-treated and placebo-treated events respectively): sensitivity to light (60.9% and 60.8%), fatigue (50.7% and 50.3%), neck pain (40.2% and 40.1%), sensitivity to sound (35.9% and 36.1%), and dizziness (29.0% and 31.0%). Between 30.8% and 57.2% of these symptoms were moderate or severe in intensity. Prodrome symptoms during the double-blind treatment period are summarized in Table 12. Prodrome symptoms identified at baseline are symptoms experienced by participants at the beginning of prodrome events.

TABLE 12
Summary of Prodrome Symptoms Identified at Baseline in the
Double-Blind Period (Modified Intent-to-Treat Population)

| | Placebo | | Ubrogepant 100 mg | |
|---|---|---|---|---|
| | (N = 449) | | (N = 448) | |
| Sensitivity to Light, n(%) | 273 | (60.8) | 273 | (60.9) |
| Mild | 156 | (57.1) | 158 | (57.9) |
| Moderate | 96 | (35.2) | 98 | (35.9) |
| Severe | 21 | (7.7) | 17 | (6.2) |
| Tired/sleepy/fatigue, n(%) | 226 | (50.3) | 227 | (50.7) |
| Mild | 108 | (47.8) | 110 | (48.5) |
| Moderate | 97 | (42.9) | 102 | (44.9) |
| Severe | 21 | (9.3) | 15 | (6.6) |
| Neck pain/stiff neck, n (%) | 180 | (40.1) | 180 | (40.2) |
| Mild | 77 | (42.8) | 80 | (44.4) |
| Moderate | 88 | (48.9) | 87 | (48.3) |
| Severe | 15 | (8.3) | 13 | (7.2) |
| Sensitivity to sound, n (%) | 162 | (36.1) | 161 | (35.9) |
| Mild | 99 | (61.1) | 103 | (64.0) |
| Moderate | 57 | (35.2) | 52 | (32.3) |
| Severe | 6 | (3.7) | 6 | (3.7) |
| Dizziness/lightheaded/ | 139 | (31.0) | 130 | (29.0) |
| vertigo/imbalance, n (%) | | | | |
| Mild | 95 | (68.3) | 90 | (69.2) |
| Moderate | 40 | (28.8) | 36 | (27.7) |
| Severe | 4 | (2.9) | 4 | (3.1) |
| Irritable, n(%) | 122 | (27.2) | 118 | (26.3) |
| Mild | 60 | (49.2) | 58 | (57.6) |
| Moderate | 51 | (41.8) | 44 | (37.3) |
| Severe | 11 | (9.0) | 6 | (5.1) |
| Nausea, n(%) | 101 | (22.5) | 106 | (23.7) |
| Mild | 75 | (74.3) | 61 | (57.5) |
| Moderate | 23 | (22.8) | 43 | (40.6) |
| Severe | 3 | (3.0) | 2 | (1.9) |

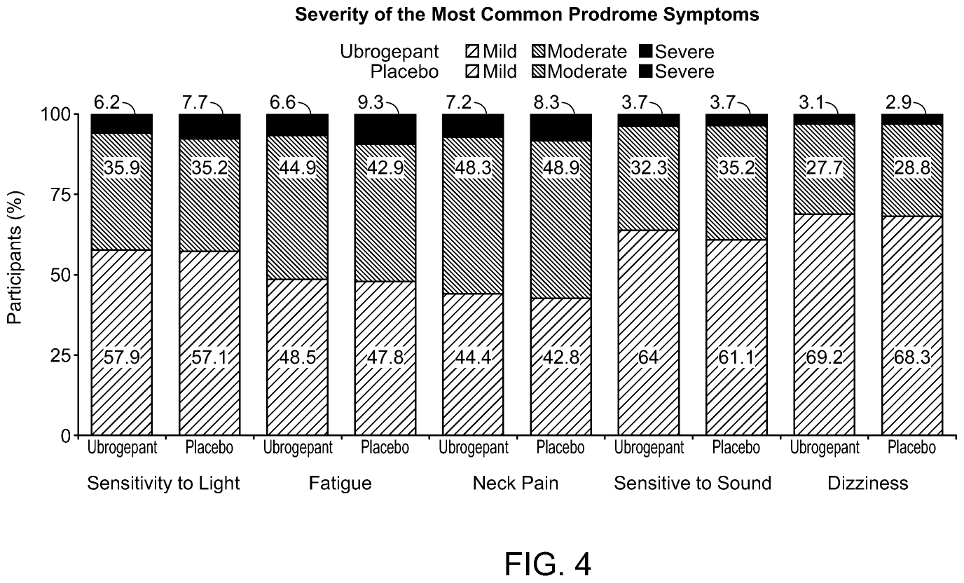

Severity of the most common prodrome symptoms is summarized in Table 13 and illustrated in FIG. 4. Of the most common prodrome symptoms reported, neck pain and fatigue had the highest rates of moderate-to-severe intensity.

TABLE 13
Severity of the Most Common Prodrome Symptoms

| | Placebo | Ubrogepant 100 mg |
|---|---|---|
| | (N = 449) | (N = 448) |
| Sensitivity to Light (%) | | |
| Mild | 57.1 | 57.9 |
| Moderate | 35.2 | 35.9 |
| Severe | 7.7 | 6.2 |
| Fatigue (%) | | |
| Mild | 47.8 | 48.5 |
| Moderate | 42.9 | 44.9 |
| Severe | 9.3 | 6.6 |
| Neck pain (%) | | |
| Mild | 42.8 | 44.4 |
| Moderate | 48.9 | 48.3 |
| Severe | 8.3 | 7.2 |
| Sensitivity to sound (%) | | |
| Mild | 61.1 | 64 |
| Moderate | 35.2 | 32.3 |
| Severe | 3.7 | 3.7 |
| Dizziness (%) | | |
| Mild | 68.3 | 69.2 |
| Moderate | 28.8 | 27.7 |
| Severe | 2.9 | 3.1 |

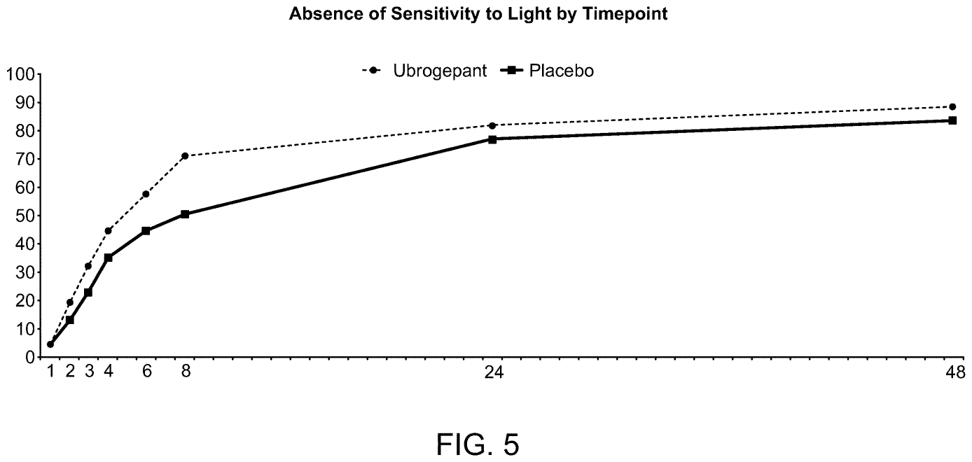

The absence of sensitivity to light over time is shown in Table 14 and in FIG. 5. N1 is the number of participants with the prodrome symptom at predose. N2 is the number of participants with non-missing prodrome symptom intensity assessment by timepoint after dose. The proportion of events with absence of sensitivity to light after ubrogepant 100 mg was numerically greater than after placebo, starting at 2 hours post-dose and extending through 48 hours (nominal P≤0.0109 for hours 2-8).

TABLE 14
Absence of Sensitivity to Light Over Time

| | | |
|---|---|---|
| | Placebo | Ubrogepant 100 mg |
| Sensitivity to light, N1 | (N1 = 273) | (N1 = 273) |

1 Hour

| N2 | 258 | | 252 | |
|---|---|---|---|---|
| Responder, n (%) | 12 | (4.7) | 12 | (4.8) |
| Nonresponder, n (%) | 246 | (95.3) | 240 | (95.2) |

| vs. placebo | | |
|---|---|---|
| Odds ratio (95% CI) | 1.08 | (0.46, 2.51) |

| P-value | 0.8594 |
|---|---|
| 2 Hours | |

| N2 | 257 | | 256 | |
|---|---|---|---|---|
| Responder, n (%) | 32 | (12.5) | 50 | (19.5) |
| Nonresponder, n (%) | 225 | (87.5) | 206 | (80.5) |

| vs. placebo | | |
|---|---|---|
| Odds ratio (95% CI) | 1.72 | (1.13, 2.61) |

| P-value | 0.0109 |
|---|---|
| 3 Hours | |

| N2 | 257 | | 250 | |
|---|---|---|---|---|
| Responder, n (%) | 60 | (23.3) | 81 | (32.4) |
| Nonresponder, n (%) | 197 | (76.7) | 169 | (67.6) |

| vs. placebo | | |
|---|---|---|
| Odds ratio (95% CI) | 1.58 | (1.12, 2.23) |

| P-value | 0.0093 |
|---|---|
| 4 Hours | |

| N2 | 249 | | 250 | |
|---|---|---|---|---|
| Responder, n (%) | 88 | (35.3) | 112 | (44.8) |
| Nonresponder, n (%) | 161 | (64.7) | 138 | (55.2) |

| vs. placebo | | |
|---|---|---|
| Odds ratio (95% CI) | 1.46 | (1.10, 1.95) |

| P-value | 0.0090 |
|---|---|
| 6 Hours | |

| N2 | 240 | | 242 | |
|---|---|---|---|---|
| Responder, n (%) | 107 | (44.6) | 140 | (57.9) |
| Nonresponder, n (%) | 133 | (55.4) | 102 | (42.1) |

| vs. placebo | | |
|---|---|---|
| Odds ratio (95% CI) | 1.75 | (1.29, 2.38) |

| P-value | 0.0004 |
|---|---|
| 8 Hours | |

| N2 | 238 | | 239 | |
|---|---|---|---|---|
| Responder, n (%) | 120 | (50.4) | 170 | (71.1) |
| Nonresponder, n (%) | 118 | (49.6) | 69 | (28.9) |

| vs. placebo | | |
|---|---|---|
| Odds ratio (95% CI) | 2.40 | (1.75, 3.27) |

| P-value | <.0001 |
|---|---|
| 24 Hours | |

| N2 | 244 | | 248 | |
|---|---|---|---|---|
| Responder, n (%) | 187 | (76.6) | 203 | (81.9) |
| Nonresponder, n (%) | 57 | (23.4) | 45 | (18.1) |

| vs. placebo | | |
|---|---|---|
| Odds ratio (95% CI) | 1.37 | (0.94, 1.99) |

| P-value | 0.1005 |
|---|---|
| 48 Hours | |

| N2 | 221 | | 220 | |
|---|---|---|---|---|
| Responder, n (%) | 184 | (83.3) | 194 | (88.2) |
| Nonresponder, n (%) | 37 | (16.7) | 26 | (11.8) |

| vs. placebo | | |
|---|---|---|
| Odds ratio (95% CI) | 1.52 | (1.00, 2.30) |

| | |
|---|---|
| P-value | 0.0490 |

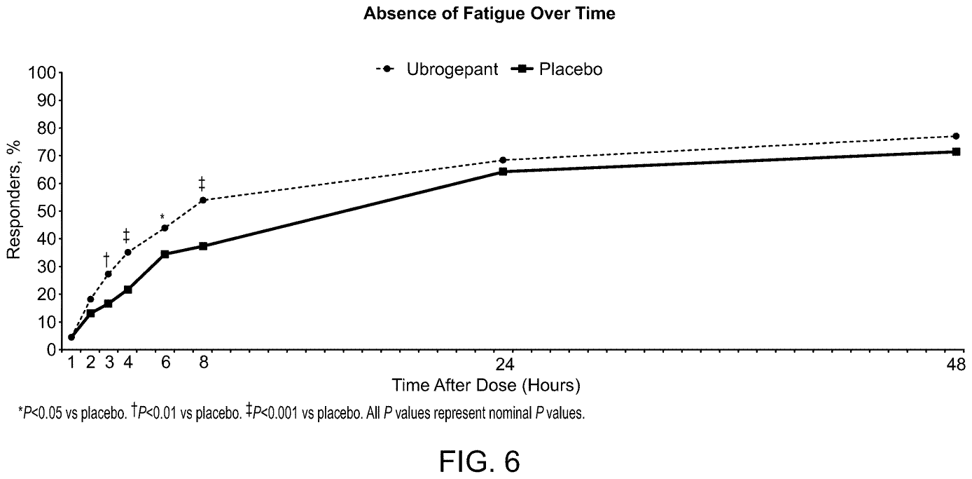

The absence of fatigue over time is shown in Table 15 and FIG. 6. N1 is the number of participants with the prodrome symptom at predose. N2 is the number of participants with non-missing prodrome symptom intensity assessment by timepoint after dose. The difference in response rates was nominally significant for the ubrogepant 100 mg dose group compared with placebo as early as 3 hours post-dose administered during the prodrome.

TABLE 15
Absence of Fatigue Over Time

| | | |
|---|---|---|
| | Placebo | Ubrogepant 100 mg |
| Fatigue, N1 | (N1 = 226) | (N1 = 227) |

1 Hour

| N2 | 218 | | 206 | |
|---|---|---|---|---|
| Responder, n (%) | 9 | (4.1) | 10 | (4.9) |
| Nonresponder, n (%) | 209 | (95.9) | 196 | (95.1) |

| vs. placebo | | |
|---|---|---|
| Odds ratio (95% CI) | 1.23 | (0.47, 3.21) |

| P-value | 0.6662 |
|---|---|
| 2 Hours | |

| N2 | 213 | | 212 | |
|---|---|---|---|---|
| Responder, n (%) | 29 | (13.6) | 39 | (18.4) |
| Nonresponder, n (%) | 184 | (86.4) | 173 | (81.6) |

| vs. placebo | | |
|---|---|---|
| Odds ratio (95% CI) | 1.43 | (0.90, 2.25) |

| P-value | 0.1264 |
|---|---|
| 3 Hours | |

| N2 | 208 | | 205 | |
|---|---|---|---|---|
| Responder, n (%) | 35 | (16.8) | 56 | (27.3) |
| Nonresponder, n (%) | 173 | (83.2) | 149 | (72.7) |
| vs. placebo | | | | |
| Odds ratio (95% CI) | | | 1.85 | (1.17, 2.92) |

| P-value | 0.0091 |
|---|---|
| 4 Hours | |

| N2 | 203 | | 207 | |
|---|---|---|---|---|
| Responder, n (%) | 44 | (21.7) | 72 | (34.8) |
| Nonresponder, n (%) | 159 | (78.3) | 135 | (65.2) |
| vs. placebo | | | | |
| Odds ratio (95% CI) | | | 1.93 | (1.36, 2.75) |

| P-value | 0.0003 |
|---|---|
| 6 Hours | |

| N2 | 195 | | 197 | |
|---|---|---|---|---|
| Responder, n (%) | 67 | (34.4) | 86 | (43.7) |
| Nonresponder, n (%) | 128 | (65.6) | 111 | (56.3) |
| vs. placebo | | | | |
| Odds ratio (95% CI) | | | 1.46 | (1.06, 2.00) |

| P-value | 0.0195 |
|---|---|
| 8 Hours | |

| N2 | 189 | | 191 | |
|---|---|---|---|---|
| Responder, n (%) | 71 | (37.6) | 103 | (53.9) |
| Nonresponder, n (%) | 118 | (62.4) | 88 | (46.1) |
| vs. placebo | | | | |
| Odds ratio (95% CI) | | | 1.87 | (1.35, 2.57) |

| P-value | 0.0002 |
|---|---|
| 24 Hours | |

| N2 | 205 | | 207 | |
|---|---|---|---|---|
| Responder, n (%) | 132 | (64.4) | 142 | (68.6) |
| Nonresponder, n (%) | 73 | (35.6) | 65 | (31.4) |
| vs. placebo | | | | |
| Odds ratio (95% CI) | | | 1.19 | (0.85, 1.66) |

| P-value | 0.3079 |
|---|---|
| 48 Hours | |

| N2 | 182 | | 179 | |
|---|---|---|---|---|
| Responder, n (%) | 130 | (71.4) | 138 | (77.1) |
| Nonresponder, n (%) | 52 | (28.6) | 41 | (22.9) |
| vs. placebo | | | | |
| Odds ratio (95% CI) | | | 1.22 | (0.84, 1.79) |

| | |
|---|---|
| P-value | 0.2944 |

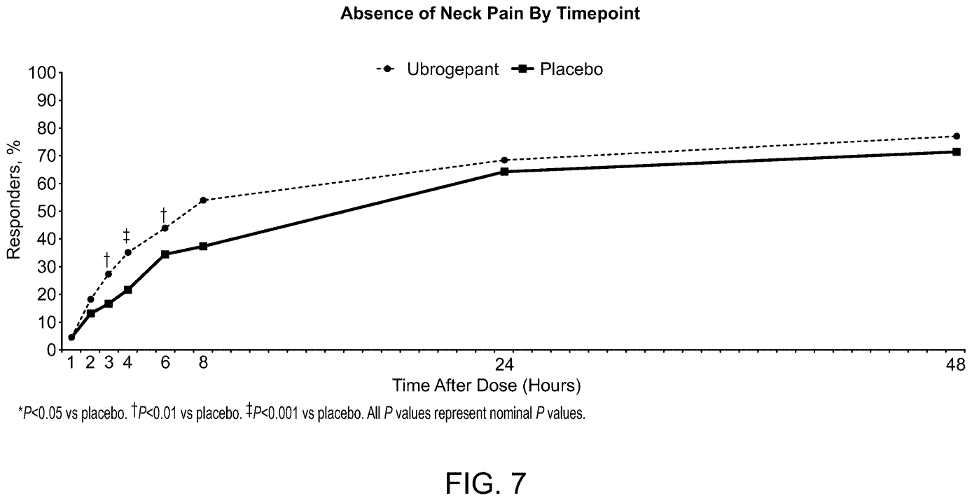

The absence of neck pain over time is shown in Table 16 and FIG. 7. N1 is the number of participants with the prodrome symptom at predose. N2 is the number of participants with non-missing prodrome symptom intensity assessment by timepoint after dose. The difference in response rates was nominally significant for the ubrogepant 100 mg dose group compared with placebo as early as 3 hours post-dose administered during the prodrome.

TABLE 16
Absence of Neck Pain Over Time

| | | |
|---|---|---|
| | Placebo | Ubrogepant 100 mg |
| Neck pain, N1 | (N1 = 180) | (N1 = 180) |

1 Hour

| N2 | 170 | | 168 | |
|---|---|---|---|---|
| Responder, n (%) | 5 | (2.9) | 7 | (4.2) |
| Nonresponder, n (%) | 165 | (97.1) | 161 | (95.8) |
| vs. placebo | | | | |
| Odds ratio (95% CI) | | | 1.43 | (0.44, 4.60) |

| P-value | 0.5478 |
|---|---|
| 2 Hours | |

| N2 | 168 | | 169 | |
|---|---|---|---|---|
| Responder, n (%) | 19 | (11.3) | 26 | (15.4) |
| Nonresponder, n (%) | 149 | (88.7) | 143 | (84.6) |
| vs. placebo | | | | |
| Odds ratio (95% CI) | | | 1.39 | (0.75, 2.56) |

| P-value | 0.2937 |
|---|---|
| 3 Hours | |

| N2 | 170 | | 159 | |
|---|---|---|---|---|
| Responder, n (%) | 27 | (15.9) | 46 | (28.9) |
| Nonresponder, n (%) | 143 | (84.1) | 113 | (71.1) |
| vs. placebo | | | | |
| Odds ratio (95% CI) | | | 2.04 | (1.25, 3.32) |

| | |
|---|---|
| P-value | 0.0045 |
| 4 Hours | |

| N2 | 169 | | 163 | |
|---|---|---|---|---|
| Responder, n (%) | 37 | (21.9) | 63 | (38.7) |
| Nonresponder, n (%) | 132 | (78.1) | 100 | (61.3) |
| vs. placebo | | | | |
| Odds ratio (95% CI) | | | 2.27 | (1.51, 3.39) |

| P-value | <0.0001 |
|---|---|
| 6 Hours | |

| N2 | 162 | | 152 | |
|---|---|---|---|---|
| Responder, n (%) | 57 | (35.2) | 73 | (48.0) |
| Nonresponder, n (%) | 105 | (64.8) | 79 | (52.0) |
| vs. placebo | | | | |
| Odds ratio (95% CI) | | | 1.68 | (1.20, 2.35) |

| P-value | 0.0026 |
|---|---|
| 8 Hours | |

| N2 | 159 | | 149 | |
|---|---|---|---|---|
| Responder, n (%) | 66 | (41.5) | 75 | (50.3) |
| Nonresponder, n (%) | 93 | (58.5) | 74 | (49.7) |
| vs. placebo | | | | |
| Odds ratio (95% CI) | | | 1.40 | (0.95, 2.06) |

| P-value | 0.0875 |
|---|---|
| 24 Hours | |

| N2 | 162 | | 168 | |
|---|---|---|---|---|
| Responder, n (%) | 103 | (63.6) | 120 | (71.4) |
| Nonresponder, n (%) | 59 | (36.4) | 48 | (28.6) |
| vs. placebo | | | | |
| Odds ratio (95% CI) | | | 1.42 | (0.96, 2.12) |

| P-value | 0.0798 |
|---|---|
| 48 Hours | |

| N2 | 153 | | 144 | |
|---|---|---|---|---|
| Responder, n (%) | 118 | (77.1) | 112 | (77.8) |
| Nonresponder, n (%) | 35 | (22.9) | 32 | (22.2) |
| vs. placebo | | | | |
| Odds ratio (95% CI) | | | 1.01 | (0.65, 1.58) |

| | |
|---|---|
| P-value | 0.9618 |

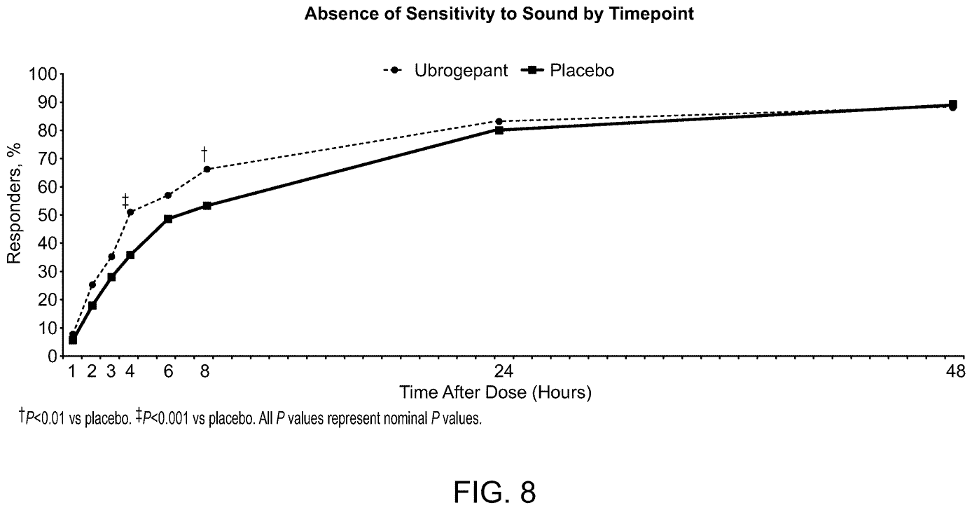

The absence of sensitivity to sound is shown in Table 17 and FIG. 8. N1 is the number of participants with the prodrome symptom at predose. N2 is the number of participants with non-missing prodrome symptom intensity assessment by timepoint after dose. Numerically higher response rates for the ubrogepant 100 mg dose group compared with placebo administered during the prodrome were observed.

TABLE 17
Absence of Sensitivity to Sound Over Time

| | | |
|---|---|---|
| Sensitivity to | Placebo | Ubrogepant 100 mg |
| sound, N1 | (N1 = 162) | (N1 = 161) |

1 Hour

| N2 | 151 | | 150 | |
|---|---|---|---|---|
| Responder, n (%) | 9 | (6.0) | 12 | (8.0) |
| Nonresponder, n (%) | 142 | (94.0) | 138 | (92.0) |
| vs. placebo | | | | |
| Odds ratio (95% CI) | | | 1.23 | (0.59, 2.57) |

| P-value | 0.5803 |
|---|---|
| 2 Hours | |

| N2 | 153 | | 154 | |
|---|---|---|---|---|
| Responder, n (%) | 27 | (17.6) | 39 | (25.3) |
| Nonresponder, n (%) | 126 | (82.4) | 115 | (74.7) |
| vs. placebo | | | | |
| Odds ratio (95% CI) | | | 1.59 | (0.99, 2.55) |

| P-value | 0.0552 |
|---|---|
| 3 Hours | |

| N2 | 149 | | 149 | |
|---|---|---|---|---|
| Responder, n (%) | 42 | (28.2) | 53 | (35.6) |
| Nonresponder, n (%) | 107 | (71.8) | 96 | (64.4) |
| vs. placebo | | | | |
| Odds ratio (95% CI) | | | 1.45 | (0.98, 2.15) |

| P-value | 0.0655 |
|---|---|
| 4 Hours | |

| N2 | 148 | | 148 | |
|---|---|---|---|---|
| Responder, n (%) | 53 | (35.8) | 75 | (50.7) |
| Nonresponder, n (%) | 95 | (64.2) | 73 | (49.3) |
| vs. placebo | | | | |
| Odds ratio (95% CI) | | | 1.97 | (1.38, 2.80) |

| P-value | 0.0002 |
|---|---|
| 6 Hours | |

| N2 | 138 | | 141 | |
|---|---|---|---|---|
| Responder, n (%) | 67 | (48.6) | 80 | (56.7) |
| Nonresponder, n (%) | 71 | (51.4) | 61 | (43.3) |
| vs. placebo | | | | |
| Odds ratio (95% CI) | | | 1.44 | (0.99, 2.11) |

| P-value | 0.0593 |
|---|---|
| 8 Hours | |

| N2 | 139 | | 145 | |
|---|---|---|---|---|
| Responder, n (%) | 74 | (53.2) | 96 | (66.2) |
| Nonresponder, n (%) | 65 | (46.8) | 49 | (33.8) |
| vs. placebo | | | | |
| Odds ratio (95% CI) | | | 1.75 | (1.17, 2.62) |

| P-value | 0.0071 |
|---|---|
| 24 Hours | |

| N2 | 143 | | 148 | |
|---|---|---|---|---|
| Responder, n (%) | 114 | (79.7) | 123 | (83.1) |
| Nonresponder, n (%) | 29 | (20.3) | 25 | (16.9) |
| vs. placebo | | | | |
| Odds ratio (95% CI) | | | 1.38 | (0.88, 2.15) |

| P-value | 0.1618 |
|---|---|
| 48 Hours | |

| N2 | 134 | | 128 | |
|---|---|---|---|---|
| Responder, n (%) | 119 | (88.8) | 113 | (88.3) |
| Nonresponder, n (%) | 15 | (11.2) | 15 | (11.7) |
| vs. placebo | | | | |
| Odds ratio (95% CI) | | | 0.93 | (0.49, 1.77) |

| | |
|---|---|
| P-value | 0.8300 |

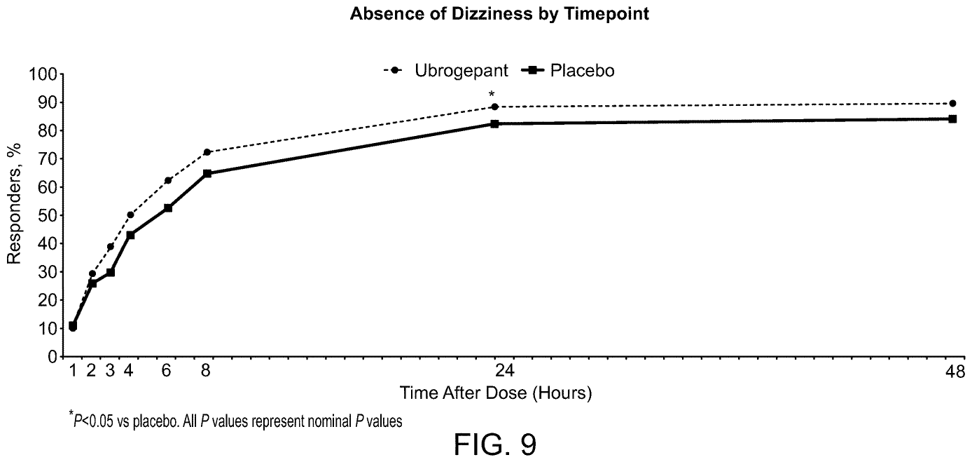

The absence of dizziness over time is shown in Table 18 and FIG. 9. N1 is the number of participants with the prodrome symptom at predose. N2 is the number of participants with non-missing prodrome symptom intensity assessment by timepoint after dose. Numerically higher response rates for the ubrogepant 100 mg dose group compared with placebo administered during the prodrome were observed.

TABLE 18
Absence of Dizziness Over Time

| | | |
|---|---|---|
| | Placebo | Ubrogepant 100 mg |
| Dizziness, N1 | (N1 = 139) | (N1 = 130) |

1 Hour

| N2 | 129 | | 120 | |
|---|---|---|---|---|
| Responder, n (%) | 14 | (10.9) | 12 | (10.0) |
| Nonresponder, n (%) | 115 | (89.1) | 108 | (90.0) |
| vs. placebo | | | | |
| Odds ratio (95% CI) | | | 0.96 | (0.45, 2.02) |

| P-value | 0.9096 |
|---|---|
| 2 Hours | |

| N2 | 127 | | 123 | |
|---|---|---|---|---|
| Responder, n (%) | 33 | (26.0) | 36 | (29.3) |
| Nonresponder, n (%) | 94 | (74.0) | 87 | (70.7) |
| vs. placebo | | | | |
| Odds ratio (95% CI) | | | 1.17 | (0.72, 1.91) |

| P-value | 0.5212 |
|---|---|
| 3 Hours | |

| N2 | 130 | | 122 | |
|---|---|---|---|---|
| Responder, n (%) | 38 | (29.2) | 48 | (39.3) |
| Nonresponder, n (%) | 92 | (70.8) | 74 | (60.7) |
| vs. placebo | | | | |
| Odds ratio (95% CI) | | | 1.60 | (0.98, 2.60) |

| P-value | 0.0607 |
|---|---|
| 4 Hours | |

| N2 | 131 | | 122 | |
|---|---|---|---|---|
| Responder, n (%) | 56 | (42.7) | 61 | (50.0) |
| Nonresponder, n (%) | 75 | (57.3) | 61 | (50.0 |
| vs. placebo | | | | |
| Odds ratio (95% CI) | | | 1.37 | (0.87, 2.15) |

| P-value | 0.1772 |
|---|---|
| 6 Hours | |

| N2 | 124 | | 119 | |
|---|---|---|---|---|
| Responder, n (%) | 65 | (52.4) | 74 | (62.2) |
| Nonresponder, n (%) | 59 | (47.6) | 45 | (37.8) |
| vs. placebo | | | | |
| Odds ratio (95% CI) | | | 1.45 | (0.96, 2.19) |

| P-value | 0.0780 |
|---|---|
| 8 Hours | |

| N2 | 124 | | 111 | |
|---|---|---|---|---|
| Responder, n (%) | 80 | (64.5) | 80 | (72.1) |
| Nonresponder, n (%) | 44 | (35.5) | 31 | (27.9) |
| vs. placebo | | | | |
| Odds ratio (95% CI) | | | 1.33 | (0.82, 2.17) |

| P-value | 0.2451 |
|---|---|
| 24 Hours | |

| N2 | 130 | | 122 | |
|---|---|---|---|---|
| Responder, n (%) | 107 | (82.3) | 108 | (88.5) |
| Nonresponder, n (%) | 23 | (17.7) | 14 | (11.5) |
| vs. placebo | | | | |
| Odds ratio (95% CI) | | | 1.82 | (1.00, 3.30) |

| P-value | 0.0489 |
|---|---|
| 48 Hours | |

| N2 | 118 | | 105 | |
|---|---|---|---|---|
| Responder, n (%) | 99 | (83.9) | 94 | (89.5) |
| Nonresponder, n (%) | 19 | (16.1) | 11 | (10.5) |
| vs. placebo | | | | |
| Odds ratio (95% CI) | | | 1.66 | (0.85, 3.24) |

| | |
|---|---|
| P-value | 0.1359 |

The most commonly reported prodrome symptoms were sensitivity to light, fatigue, neck pain, sensitivity to sound, and dizziness. Between 30.8% and 57.2% of these common prodrome symptoms were moderate or severe in intensity. Treatment with ubrogepant 100 mg during the prodrome ameliorated the most commonly reported prodrome symptoms compared with placebo. This efficacy data was collected in all participants in the mITT population and included those who did and did not develop a headache.

The absence of aura at various timepoints post dose in the mITT population is shown in Table 19. N₁ is the number of patients with non-missing aura presence assessment at timepoint.

TABLE 19
Absence of Aura at Various Timepoints
Post Dose (mITT Population)

| | Placebo | | Ubrogepant 100 mg | |
|---|---|---|---|---|
| Statistics at Hours Post Dose | (N = 449) | | (N = 448) | |
| Predose, N₁ | 251 | | 247 | |
| Absence of aura, n (%) | 127 | (50.6) | 125 | (50.6) |
| Presence of aura, n (%) | 124 | (49.4) | 122 | (49.4) |
| 1 Hour, N₁ | 239 | | 230 | |
| Responder, n (%) | 149 | (62.3) | 142 | (61.7) |
| Nonresponder, n (%) | 90 | (37.7) | 88 | (38.3) |
| vs placebo, OR (95% CI) | | | 0.99 | (0.68, 1.42) |
| P-value | | | 0.9398 | |
| 2 Hours, N₁ | 238 | | 235 | |
| Responder, n (%) | 164 | (68.9) | 169 | (71.9) |
| Nonresponder, n (%) | 74 | (31.1) | 66 | (28.1) |
| vs placebo, OR (95% CI) | | | 1.13 | (0.84, 1.54) |
| P-value | | | 0.4152 | |
| 3 Hours, N₁ | 237 | | 226 | |
| Responder, n (%) | 181 | (76.4) | 174 | (77.0) |
| Nonresponder, n (%) | 56 | (23.6) | 52 | (23.0) |
| vs placebo, OR (95% CI) | | | 0.96 | (0.69, 1.35) |
| P-value | | | 0.8354 | |
| 4 Hours, N₁ | 235 | | 225 | |
| Responder, n (%) | 187 | (79.6) | 182 | (80.9) |
| Nonresponder, n (%) | 48 | (20.4) | 43 | (19.1) |
| vs placebo, OR (95% CI) | | | 1.12 | (0.80, 1.56) |
| P-value | | | 0.5206 | |
| 6 Hours, N₁ | 224 | | 216 | |
| Responder, n (%) | 187 | (83.5) | 183 | (84.7) |
| Nonresponder, n (%) | 37 | (16.5) | 33 | (15.3) |
| vs placebo, OR (95% CI) | | | 1.05 | (0.68, 1.61) |
| P-value | | | 0.8373 | |
| 8 Hours, N₁ | 217 | | 210 | |
| Responder, n (%) | 190 | (87.6) | 188 | (89.5) |
| Nonresponder, n (%) | 27 | (12.4) | 22 | (10.5) |
| vs placebo, OR (95% CI) | | | 1.10 | (0.65, 1.85) |
| P-value | | | 0.7291 | |
| 24 Hours, N₁ | 221 | | 228 | |
| Responder, n (%) | 203 | (91.9) | 216 | (94.7) |
| Nonresponder, n (%) | 18 | (8.1) | 12 | (5.3) |
| vs placebo, OR (95% CI) | | | 1.48 | (0.83, 2.65) |
| P-value | | | 0.1810 | |
| 48 Hours, N₁ | 202 | | 195 | |
| Responder, n (%) | 187 | (92.6) | 190 | (97.4) |
| Nonresponder, n (%) | 15 | (7.4) | 5 | (2.6) |
| vs placebo, OR (95% CI) | | | 3.05 | (1.18, 7.85) |

| | |
|---|---|
| P-value | 0.0215 |

N₁ = Number of patients with non-missing aura presence assessment at timepoint.
OR (95% CI) and P-value are based on GLMM with treatment group, treatment period, and predose baseline aura status as categorical fixed effects. An unstructured covariance matrix is selected for the covariance matrix of the residual effect for the repeated measurements (corresponding to the 2 qualifying prodrome events) within a patient. Covariance structure of compound symmetry is used when model does not converge.
Source: Data on file, 304-002.

Safety results were generally consistent with the known safety profile of ubrogepant from previous ubrogepant studies.

Tables 20 and 21 provide an overall summary of adverse events during the double-blind treatment period for the safety population.

TABLE 20
Overview of Adverse Events within 48 Hours Following Administration
of Study Intervention - Safety Population

| | | | Ubrogepant | | | |
|---|---|---|---|---|---|---|
| | Placebo | | 100 mg | | Total | |
| | (N = 462) | | (N = 456) | | (N = 480) | |
| | n (%) | | n (%) | | n (%) | |
| Treatment-emergent | 55 | (11.9) | 77 | (16.9) | 112 | (23.3) |
| adverse events (TEAE) | | | | | | |
| Treatment-related TEAEs | 42 | (9.1) | 60 | (13.2) | 89 | (18.5) |
| Treatment-emergent | 0 | (0.0) | 0 | (0.0) | 0 | (0.0) |
| Serious adverse | | | | | | |
| events (TESAE) | | | | | | |
| Treatment-related TESAEs | 0 | (0.0) | 0 | (0.0) | 0 | (0.0) |
| Death | 0 | (0.0) | 0 | (0.0) | 0 | (0.0) |
| Adverse event leading to | 0 | (0.0) | 0 | (0.0) | 0 | (0.0) |
| discontinuation (ADO) | | | | | | |

TABLE 21
Overview of Adverse Events within 30 Days Following Administration
of Study Intervention - Safety Population

| | | | Ubrogepant | | | |
|---|---|---|---|---|---|---|
| | Placebo | | 100 mg | | Total | |
| | (N = 462) | | (N = 456) | | (N = 480) | |
| | n (%) | | n (%) | | n (%) | |
| Treatment-emergent | 72 | (15.6) | 90 | (19.7) | 137 | (28.5) |
| adverse events (TEAE) | | | | | | |
| Treatment-related TEAEs | 45 | (9.7) | 60 | (13.2) | 92 | (19.2) |
| Treatment-emergent | 1 | (0.2) | 0 | (0.0) | 1 | (0.2) |
| Serious adverse | | | | | | |
| events (TESAE) | | | | | | |
| Treatment-related TESAEs | 1 | (0.2) | 0 | (0.0) | 1 | (0.2) |
| Death | 0 | (0.0) | 0 | (0.0) | 0 | (0.0) |
| Adverse event leading to | 0 | (0.0) | 0 | (0.0) | 0 | (0.0) |
| discontinuation (ADO) | | | | | | |

The most common TEAEs (≥2% in either treatment group) within 48 hours following administration of study intervention were nausea (placebo 15 of 462 [3.2%], ubrogepant 23 of 456 [5.0%]); dizziness (placebo 12 of 462 [2.6%], ubrogepant 11 of 456 [2.4%]), fatigue (placebo 7 of 462 [1.5%], ubrogepant 12 of 456 [2.6%]) and somnolence (placebo 5 of 462 [1.1%], ubrogepant 11 of 456 [2.4%]). Nausea (placebo 3.7%, ubrogepant 5.3%) was also the most common TEAE within 30 days following administration of study intervention. Treatment emergent adverse events that occurred in ≥2% participants in either treatment group are presented in Tables 22 and 23.

TABLE 22
Treatment Emergent Adverse Events Reported by At Least 2% of
Participants in Either Treatment Group Within 48 Hours Following
Administration of Study Medication - Safety Population

| | Placebo | | Ubrogepant 100 | | Total | |
|---|---|---|---|---|---|---|
| | (N = 462) | | mg (N = 456) | | (N = 480) | |
| | n (%) | | n (%) | | n (%) | |
| Nausea | 15 | (3.2) | 23 | (5.0) | 36 | (7.5) |
| Dizziness | 12 | (2.6) | 11 | (2.4) | 19 | (4.0) |
| Fatigue | 7 | (1.5) | 12 | (2.6) | 16 | (3.3) |
| Somnolence | 5 | (1.1) | 11 | (2.4) | 16 | (3.3) |

TABLE 23
Treatment Emergent Adverse Events Reported by At Least 2% of
Participants in Either Treatment Group Within 30 Days Following
Administration of Study Medication - Safety Population

| | Placebo | | Ubrogepant 100 | | Total | |
|---|---|---|---|---|---|---|
| | (N = 462) | | mg (N = 456) | | (N = 480) | |
| | n (%) | | n (%) | | n (%) | |
| Nausea | 17 | (3.7) | 24 | (5.3) | 38 | (7.9) |
| Dizziness | 14 | (3.0) | 11 | (2.4) | 21 | (4.4) |
| Fatigue | 7 | (1.5) | 12 | (2.6) | 16 | (3.3) |
| Somnolence | 5 | (1.1) | 11 | (2.4) | 16 | (3.3) |

One participant in ubrogepant 100 mg/placebo sequence had 1 TESAE (treatment-emergent serious adverse event). The TESAE was rhabdomyolysis and was reported on Day 6 of Period 2.

No participant was discontinued during the DB treatment period due to adverse events in the safety population.

The results of the study show that treatment with ubrogepant during the prodrome (or premonitory phase), prior to the onset of headache, prevents the development of moderate/severe headache, or headache of any intensity, over the next 24 hours. Ubrogepant was superior to placebo in reducing the development of moderate/severe headache within the 24 and 48 hours after administration, as well as the development of headache of any intensity within 24-hours post dose. In addition, when administered during the prodrome phase of migraine, ubrogepant improves the ability to function normally over the next 24 hours. The treatment was well-tolerated; nausea, fatigue, dizziness, and somnolence were reported by ≤5% of participants. The study challenges the generally accepted recommendation to treat migraine attacks at the first sign of headache pain, suggesting that acute treatment with ubrogepant administered prior to headache phase onset, during the prodrome phase, can prevent or minimize headache occurrence.

1. A method of treating migraine, the method comprising administering a CGRP receptor antagonist to a patient in need thereof, wherein the CGRP receptor antagonist is administered during the prodrome stage of a migraine attack.

2. The method according to claim 1, wherein the CGRP receptor antagonist is selected from the group consisting of ubrogepant, atogepant, rimegepant, zavegepant, or a pharmaceutically acceptable salt thereof.

3. The method according to claim 2, wherein the CGRP receptor antagonist is ubrogepant or a pharmaceutically acceptable salt thereof.

4. The method according to claim 3, wherein ubrogepant is administered in an amount selected from 50 mg or 100 mg.

5. The method according to claim 4, wherein a second dose of ubrogepant is administered at least two hours after the initial dose of ubrogepant.

6. The method according to claims 1-5, wherein ubrogepant is administered when a patient is experiencing at least one symptom of prodrome selected from the group consisting of cognitive and mood changes, homeostatic and hormonal changes, migrainous and sensory sensitivities, and cranial autonomic symptoms.

7. The method according to claims 1-6, wherein treatment with the CGRP receptor antagonist reduces or eliminates migraine headache in the patient.

8. The method according to claims 1-7, wherein treatment with the CGRP receptor antagonist reduces or eliminates at least one symptom selected from the group consisting of pain, aura, photophobia, phonophobia, fatigue, neck pain, and dizziness.

9. A method of treating migraine, the method comprising administering ubrogepant or a pharmaceutically acceptable salt thereof to a patient in need thereof, wherein ubrogepant is administered during the prodrome stage of the migraine.

10. The method according to claim 9, wherein ubrogepant is administered at a dose selected from 50 mg and 100 mg.

11. The method according to claim 10, wherein ubrogepant is administered at a dose of 50 mg.

12. The method according to claim 10, wherein ubrogepant is administered at a dose of 100 mg.

13. The method according to claims 9-12, wherein treatment with ubrogepant during the prodrome stage of a migraine attack results in a statistically significant treatment effect with respect to the absence of a headache of moderate or severe intensity within 24 hours of administration as compared to placebo.

14. The method according to claims 9-13, wherein treatment with ubrogepant during the prodrome stage of a migraine attack results in a statistically significant treatment effect with respect to the absence of a headache of moderate or severe intensity within 48 hours of administration as compared to placebo.

15. The method according to claims 9-14, wherein treatment with ubrogepant during the prodrome stage of a migraine attack results in a statistically significant treatment effect with respect to the absence of a headache of any intensity within 24 hours of administration as compared to placebo.

16. The method according to claims 9-15, wherein ubrogepant is administered when a patient is experiencing at least one symptom of prodrome selected from the group consisting of cognitive and mood changes, homeostatic and hormonal changes, migrainous and sensory sensitivities, and cranial autonomic symptoms.

17. The method according to claims 9-16, wherein treatment with the CGRP receptor antagonist reduces or eliminates at least one symptom selected from the group consisting of pain, aura, photophobia, phonophobia, fatigue, neck pain, and dizziness.

18. A method for the acute treatment of migraine, the method comprising administering ubrogepant or a pharmaceutically acceptable salt thereof to a patient in need thereof, wherein the patient is experiencing a migraine but has not yet developed a migraine headache.

19. The method according to claim 18, wherein ubrogepant is administered during the prodrome stage of the migraine attack.

20. The method according to claims 18 and 19, wherein ubrogepant is administered at a dose of 50 mg or 100 mg.

21. The method according to claims 18-20, wherein treatment with ubrogepant prevents the development of a moderate or severe headache within 24 hours of administration of ubrogepant.

22. The method according to claims 18-21, wherein treatment with ubrogepant prevents the development of a headache of any intensity within 24 hours of administration of ubrogepant.
